(19) United States
(12) Patent Application Publication
(10) Pub. No.: US 2014/0234308 A1
(43) Pub. Date: Aug. 21, 2014

(54) TREATMENT OF BREAST CANCER WITH COMPANION DIAGNOSTIC

(71) Applicant: THE REGENTS OF THE UNIVERSITY OF CALIFORNIA, Oakland, CA (US)

(72) Inventors: Madhuri Wadehra, Manhattan Beach, CA (US); Jonathan Braun, Tarzana, CA (US)

(73) Assignee: The Regents of the University of California, Oakland, CA (US)

(21) Appl. No.: 14/351,825

(22) PCT Filed: Oct. 15, 2012
(86) PCT No.: PCT/US2012/060288

§ 371 Date: Apr. 14, 2014

Related U.S. Application Data
(60) Provisional application No. 61/546,982, filed on Oct. 13, 2011.

(51) Int. Cl. C07K 16/30; A61K 45/06; A61K 47/48; A61K 39/395

(52) U.S. Cl. CPC C07K 16/3015; A61K 39/39558; A61K 45/06; A61K 47/48584
USPC 424/133.1; 424/139.1; 424/183.1

(57) ABSTRACT

Despite advances in therapy, breast cancer remains the most common malignancy in women. Of particular concern is the aggressive triple negative subtype that lacks the BRCA1 mutation, estrogen receptor, and epidermal growth factor type 2 receptor (Her-2/neu), which accounts for approximately half of all breast cancer deaths. Provided herein are compositions and methods for treating breast cancer, including the triple negative subtype.

### CROSS-REFERENCES TO RELATED APPLICATIONS

This application claimed priority benefit of U.S. patent application Ser. No. 61/546,982 filed Oct. 13, 2011, the disclosure of which is incorporated by reference in its entirety.

### STATEMENT AS TO RIGHTS TO INVENTIONS MADE UNDER FEDERALLY SPONSORED RESEARCH AND DEVELOPMENT

This invention was made with Government support of Grant No. CA131756, awarded by the National Institutes of Health. The Government has certain rights in this invention.

### FIELD OF THE INVENTION

This invention relates to anti-EMP2 antibodies, their pharmaceutical compositions and methods for using them in the detection and treatment of cancers that overexpress EMP2, such as triple negative breast cancers.

### BACKGROUND OF THE INVENTION

Breast cancer remains the most common malignancy among women worldwide. Breast cancer is a heterogeneous disease, which exhibits a wide range of clinical behaviors, prognoses, and histologies (Tavassoli F, Devilee P, editors. (2003) WHO Classification of Tumors. Pathology & Genetics: Tumors of the breast and female genital organs. Lyon (France): IARC Pres). Breast cancer is the abnormal growth of cells that line the breast tissue ducts and lobules and is classified by whether the cancer started in the ducts or the lobules and whether the cells have invaded (grown or spread) through the duct or lobule, and by the way the cells look under the microscope (tissue histology). It is not unusual for a single breast tumor to have a mixture of invasive and in situ cancer.

Molecular classification of breast cancer has identified specific subtypes, often called “intrinsic” subtypes, with clinical and biological implications, including an intrinsic luminal subtype, an intrinsic HER2-enriched subtype (also referred to as the HER2⁺ or ER⁻/HER2⁺ subtype) and an intrinsic basal-like breast cancer (BLBC) subtype. (Perou et al. 2000). Identification of the intrinsic subtypes has typically been accomplished by a combination of methods, including (1) histopathological detection, (2) estrogen receptor (ER), progesterone receptor (PR) and human epidermal growth factor receptor 2 (HER2) expression status and (3) detection of characteristic cellular markers.

Basal-like breast cancer (BLBC), which expresses genes characteristic of basal epithelial cells in the normal mammary gland, comprises up to 15%-25% of all breast cancers (Kreike et al. 2007) and is associated with the worst prognosis of all breast cancer types. BLBCs underexpress estrogen receptor (ER), progesterone receptor (PR), and human epidermal growth factor receptor 2 (HER2) and encompass 60% to 90% of so-called “triple negative” (ER⁻/PR⁻/HER2) breast cancers. Although most basal-like breast cancers are often referred to as triple negative based on the expression status of ER, PR and HER2, not all basal-like breast cancers are triple negative.

Thus, the intrinsic basal-like breast cancer subtype may be further subdivided into at least three distinct subtypes described herein as “hybrid” basal-like breast cancer subtypes. In addition to a hybrid triple negative subtype, the hybrid basal-like breast cancer subtypes have profiles that resemble both basal-like breast cancer and at least one other breast cancer molecular subtype. For example, hybrid basal-like subtypes can include a hybrid basal-like/HER2⁺ subtype that has a receptor profile of ER⁻/PR⁻/HER⁺, a hybrid basal-like/luminal subtype that has a receptor profile of ER⁺/PR^{- or +/HER}⁻ or +, and a hybrid basal-like/triple negative subtype that has a receptor profile of ER⁻/PR⁻/HER⁻.

The intrinsic luminal breast cancer subtype is characterized by expression or overexpression of ER and/or PR (ER⁺ and/or PR⁺). The luminal subtype can be further subdivided based on HER2 status into the luminal A subtype, which is additionally characterized by underexpression of HER2 (ER⁺/PR^{+ or -}/HER⁻), and luminal B subtype, which is additionally characterized by overexpression of HER2 (ER⁺/PR^{+ or -}/HER⁺). Intrinsic luminal subtypes are often considered to be the most treatable breast cancer subtype and are associated with the best prognosis.

Whereas ER and HER2 guide treatment of luminal and HER2 breast cancers, respectively, chemotherapy remains the only modality of systemic therapy for BLBC. Preferentially affecting younger women, particularly African American women, BLBCs are associated with high histologic grade, aggressive clinical behavior, and a high rate of metastasis to the brain and lung (Carey et al. 2006). Unlike other breast cancer subtypes, there seems to be no correlation between tumor size and lymph node metastasis in BLBCs (Dent et al. 2007).

BLBCs are associated with expression of basal cytokeratins (CK5/6, CK14, and CK17), epidermal growth factor receptor (EGFR), c-kit, and p53 and associated with the absence of ER, PR, and HER2 expression. With a large variety of associated genes, BLBCs have been defined differently in different studies using a set of diagnostic markers. For example, Nielsen et al. defined BLBC on the basis of negative ER and negative HER2 expression in addition to positive basal cytokeratin, EGFR, and/or c-kit expression (Nielsen et al. 2004). On the other hand, other groups have defined BLBC on the basis of on a combination of negative ER, and negative HER2 expression and positive CK5, P-cadherin, and p63 expression (Elsheikh et al. 2008) or positive vimentin, EGFR, and CK5/6 expression (Livasy et al. 2006). These different technical approaches in combination with widely varying patient cohorts may explain the inconsistent experimental results for these markers.

Identification of the basal-like subtype using immunohistochemistry (IHC) for detecting hormone receptors alone is less desirable than detecting a theranostic biomarker, because identification is based on the absence of IHC staining for estrogen receptor (ER), progesterone receptor (PR), and human epidermal growth factor receptor 2 (HER2) rather than the presence of a specific tumor marker or markers. Its diagnosis is more one of exclusion rather than inclusion.

Important molecular characterizations have been made over the last decade which assist in prediction and treatment of breast cancer. These include BRCA1 mutation status as well as the expression of the estrogen receptor (ER) and epidermal growth factor type 2 receptor (ERBB2 or Her-2/neu). Gene expression profiling has recently identified specific breast cancer subgroups, each with a distinct molecular signature. This molecular classification system divides breast cancers into four biologically different subtypes: 1) normal/non-cancerous; 2) estrogen-receptor (ER)-positive luminal breast cancer; 3) ER-negative, HER2/neu overexpressing breast cancer; and 4) ER-negative breast cancer, with expression of basal-type cytokeratins (CK 5/6, 14, 17), p53, and overexpression of epidermal growth factor receptor (EGFR).

Treatment with Her-2/neu and ER modulators have shown clinical promise for tumors expressing these receptors. However, only approximately 15% of individuals have tumors with Her-2/neu over-expression thereby limiting the utility of this treatment in the general population. Furthermore, while 70% of breast cancers express ER, loss of expression and/or resistance to anti-ER therapies is a major issue.

A particularly aggressive type of breast cancer lacks expression of ER, Her-2/neu and the progesterone receptor (PR). This aggressive type of breast cancer is often referred to as triple negative breast cancer (TNBC). While this variant occurs in a minority of patients (10-15%), it accounts for approximately half of all breast cancer deaths.

Triple negative breast cancers are typically observed in young African American women and Caucasian women who carry a mutation in the BRCA1 gene. Triple negative breast cancers are typically ER/PR(−/−), Her2/neu(−/−), EGFR(+), p53(−) and cytokeratin 5/6 (+I+).

While some triple negative breast cancers respond to chemotherapy, a subset of triple negative breast cancers are chemotherapy-resistant and highly metastatic, carrying with it an extremely poor prognosis. Therefore, identifying new molecular targets expressed in this aggressive subtype is of high priority remains a need in the art.

As reported herein, the epithelial membrane protein-2 (EMP2) is overexpressed in triple negative breast cancers. EMP2 is a tetraspan protein belonging to the growth arrest specific-3 (GAS3) family. Functionally, EMP2 associates with and modulates the localization and activity of both integrin αvβ3 and focal adhesion kinase (FAK). EMP2 (SEQ ID NO:1) is expressed at high levels in epithelial cells of the lung, eye, and genitourinary tracts. Like several tetraspan proteins (CD9, CD81, PMP22), EMP2 in murine fibroblasts is localized to lipid raft domains. EMP2 controls cell surface trafficking and function of certain integrins, GPI-linked proteins, and class I MHC molecules, and reciprocally regulates caveolin expression. (see, Claas et al., J Biol Chem 276:7974-84 (2001); Hasse et al., J Neurosci Res 69:227-32 (2002); Wadehra et al., Exp Mol Pathol 74:106-12 (2003); Wadehra et al., Mol Biol Cell 15:2073-2083 (2004); Wadehra et al., J Biol Chem 277:41094-41100 (2002); and Wadehra et al., Clin Immunol 107:129-136 (2003)).

SEQ ID NO: 1
(ACCESSION P54851) MLVLLAFIIA FHITSAALLF
IATVDNAWWV GDEFFADVWR ICTNNTNCTV INDSFQEYST
LQAVQATMIL STILCCIAFF IFVLQLFRLK QGERFVLTSI
IQLMSCLCVM IAASIYTDRR EDIHDKNAKF YPVTREGSYG
YSYILAWVAF ACTFISGMMY LILRKRK

EMP2 appears to regulate trafficking of various proteins and glycolipids by facilitating transfer of molecules from post-Golgi endosomal compartments to appropriate plasma membrane locations. Specifically, EMP2 is thought to facilitate the appropriate trafficking of select molecules into glycolipids-enriched lipid raft microdomains (GEMs) (Wadehra et al., Mol Biol Cell 15:2073-83 (2004)). GEMs are cholesterol rich microdomains which are often associated with chaperones, receptosomes, and protein complexes that are important for efficient signal transduction (Leitinger et al., J Cell Sci 115:963-72 (2002); Moffett et al., J Biol Chem 275:2191-8 (2000)). Moreover, GEMs are involved in correct sorting of proteins from the Golgi apparatus to plasma membrane (Abrami et al., J Biol Chem 276:30729-36 (2001); Galbiati et al., Cell 106:403-11 (2001); Gruenberg et al., Curr Opin Cell Biol 7: 552-63 (1995)). In this respect, modulation of EMP2 expression levels or its location on the plasma membrane alters the surface repertoire of several classes of molecules including integrins, focal adhesion kinase, class I major histocompatibility molecules and other immunoglobulin super-family members such as CD54 and GPI-linked proteins (Wadehra et al., Dev Bio1287:336-45 (2005); Wadehra et al., Clinical Immunology 107:129-36 (2003); Morales et al., Invest Opthalmol Vis Sci (2008)).

EMP2 expression is associated with EMP2 neoplasia (Wadehra et al., Cancer 107:90-8 (2006)). In endometrial cancer, for example, EMP2 is an independent prognostic indicator for tumors with poor clinical outcome. EMP2 positive tumors, compared to EMP2 negative tumors, had a significantly greater myometrial invasiveness, higher clinical state, recurrent or persistent disease following surgical excision, and earlier mortality.

Based on studies described herein it is now shown that EMP2 can be used as a target in the treatment of triple negative breast cancer. Accordingly, EMP2 polypeptides, anti-EMP2 antibodies, and EMP2 siRNA can be used to diagnose and treat triple negative breast cancers. As discussed above, there remains a large need for methods and compositions which are useful in the prevention, treatment, and modulation of triple negative breast cancers. Accordingly, this invention provides novel compositions and methods for meeting these and other needs.

### BRIEF SUMMARY OF THE INVENTION

In one embodiment, the invention relates to a method of treating a patient for breast cancer. In a specific embodiment, the breast cancer is a triple negative breast cancer tumor that overexpresses EMP2. In a specific embodiment, the method comprises administering to a patient suffering from a triple negative breast cancer tumor that overexpresses EMP2 an effective amount of an anti-EMP2 antibody. In a specific embodiment, the anti-EMP2 antibody specifically binds to an epitope in the second extracellular loop of EMP2. In a specific embodiment, the epitope comprises the amino acid sequence DIHDKNAKFYPVTREGSYG. In a specific embodiment, the anti-EMP2 antibody is administered in a physiologically acceptable carrier or a pharmaceutically acceptable carrier.

In one embodiment, the anti-EMP2 antibody competes with an antibody comprising the heavy and light chain variable regions of a KS49, a KS41, a KS83, or a KS89 diabody.

In one embodiment, the anti-EMP2 antibody shares 90% amino acid identity with heavy and light chain variable regions of a KS49, a KS41, a KS83, or a KS89 diabody.

In one embodiment, the anti-EMP2 antibody comprises CDR sequences identical to those of a KS49, a KS41, a KS83, or a KS89 diabody.

In one embodiment, the method of treating a patient for breast cancer with an anti-EMP2 antibody further comprises administering to the patient an effective amount of at least one additional anti-cancer agent. In a specific embodiment, at least one additional anti-cancer agent is selected from the group consisting of platinum-based chemotherapy drugs, taxanes, tyrosine kinase inhibitors, anti-EGFR antibodies, anti-ErbB2 antibodies, and combinations thereof

In a specific embodiment, at least one additional anti-cancer agent is an EGFR inhibitor. In a specific embodiment, the EGFR inhibitor is an anti-EGFR antibody. In a specific embodiment, the anti-EGFR antibody is cetuximab. In a specific embodiment, the anti-EGFR antibody is selected from the group consisting of matuzumab, panitumumab, and nimotuzumab.

In one embodiment, the EGFR inhibitor is a small molecule inhibitor of EGFR signaling. In a specific embodiment, the small molecule inhibitor of EGFR signaling is selected from the group consisting of gefitinib, lapatinib, canertinib, pelitinib, erlotinib HCL, PKI-166, PD158780, and AG 1478.

In one embodiment, at least one additional anti-cancer agent is a VEGF inhibitor. In a specific embodiment, the VEGF inhibitor comprises an anti-VEGF antibody. In a specific embodiment, the anti-VEGF antibody is bevacizumab.

In any of the embodiments, the tissue, cancer, subject, or patient to be treated is human or mammalian.

In one embodiment, the method further comprises a companion diagnostic.

In still further embodiments, the invention also provides EMP2 polypeptides, anti-EMP2 antibodies, and EMP2 siRNA which would be of use in treating or preventing cancers which overexpress EMP2.

In certain embodiments, the EMP2 antibodies may be used in diagnosis, prognosis, or the treatment of a cancer alone or when conjugated with an effector moiety. EMP2 antibodies conjugated with toxic agents, such as ricin, as well as unconjugated antibodies, may be useful therapeutic agents naturally targeted to EMP2 bearing cancer cells. Such antibodies can be useful in blocking invasiveness. EMP polypeptides and nucleic acids may be used in vaccine therapies for the cancer.

### BRIEF DESCRIPTION OF THE DRAWINGS

FIG. 1 depicts EMP2 expression is stratified by histologic type and stage. (A) Western blot analysis was performed on whole tissue homogenates from normal and tumor regions of the breast. EMP2 (18 kD) is highly expressed in the breast tumor. Weak expression was visualized in one normal patient. (B) Normal and breast tumor tissue were stained for EMP2 expression using a tissue array. A representative patient with high EMP2 staining within a tumor is shown. (C) The mean integrated intensity of EMP2 protein expression for each category is shown using bar plots. The error bars represent the standard error of the mean; n is number of sample. EMP2 expression was significantly increased in ductal carcinoma in situ (DCIS, P<0.01), invasive ductal carcinoma (IDC, P<0.01), and metastasis (P<0.01) compared to normal. Numbers represent the number of spots analyzed. (D) Expression of EMP2 in a panel of breast cancer cells was evaluated by Western blot analysis. HS578/EMP2 cells were generated to stably overexpress EMP2. (E) Expression of EMP2 was evaluated in the murine mammary cell D2F2, cells which overxpress EMP2 (D2F2/EMP2), as well in a representative murine spleen homogenate.

FIG. 2 depicts reduced cellular invasion in the presence of anti-EMP2 IgG1 antibodies and diabodies. (A) 60 μg/m1 anti-EMP2 IgG1 or control IgG or (B) 20 μg/m1 EMP2 diabody KS83 or control diabody A10 were added to cells for 1 hr. Invasion was measured as the number of cells that migrated through the transwell. Top panel, Cells were visualized using crystal violet. Bottom panel, Averaged results from 3 experiments. (C) 60 μg/m1 anti-EMP2 IgG1 or control IgG was added to MDA-MB-468 cells for 1 hr. Alternatively, 20 ug/ml EMP2 KS83 or control diabody were added. Cells were then plated for 12 hrs and lysed. Proteins were separated by SDS-PAGE and probed using the indicated antibodies. The experiment was repeated three times, and representative image is shown. (D) pSRC expression from three experiments were quantified using Image J, and the data was normalized relative to total Src levels. Treatment with anti-EMP2 IgG1 or KS83 significantly reduced pSRC expression. Values are averages (±SEM).

FIG. 3 depicts the induction of cytostasis in the presence of Anti-EMP2 IgG1 antibodies and diabodies. (A) HS578 cells do not express any endogenous EMP2 expression, and HS578/EMP2 cells were engineered to overexpress EMP2. 60 μg/ml anti-EMP2 IgG1 or control IgG; 20 μg/ml EMP2 diabody KS83 or control diabody were added to the above cells for 72 hours. Cellular viability was determined using trypan blue exclusion, and values represent results from 3 independent experiments (±SEM). (B) ZR-75-1 and MBA-MD-468 cells were treated as above to determine cellular viability. Values are averages (±SEM, n=3). (C) EMP2 diabodies and IgG1 promote apoptosis. ZR-75-1 (top panels) and MBA-MD-468 (bottom panels) cells were incubated with 20 μg/mL KS83, 20 μg/mL control diabody, 60 μg/m1 anti-EMP2 IgG1, or 60 μg/mL control IgG. Cells were washed and stained with Annexin V and propidium iodide. Staining is expressed as the % Annexin V-7-aminoactinomycin D-positive cells above the isotype control. The experiment was repeated three times with similar results. A representative graph is shown.

FIG. 4 depicts that the targeting EMP2 reduces tumor load. (A) MDA-MB-468 cells were injected s.c. into nude BALB/c female mice. When tumors were detectable, they were injected i.t. with molar equivalent amounts of diabody (1 mg/kg), IgG1 antibody (3 mg/kg), or the indicated controls (Day 0). Mice were injected twice a week, and tumor volume was monitored using calipers. n=6. *, p<0.05 for both KS83 and anti-EMP2 IgG1 treatment compared with diabody A10 and sterile saline, respectively. (B) At day 18, tumors were excised, formalin fixed, and paraffin embedded. Tumor histology was assessed by hemotoxylin and eosin staining A representative image is shown. Magnification: 20×. Scale bar=100 μm. (C) MDA-MB-468 cells were injected as above into nude BALB/c mice as above. Mice were systemically treated with 5mg/kg anti-EMP2 IgG1 or control IgG every week. Tumor volume was determined using calipers, and values represent averages (±SEM, n=6). Comparison by Student's t-test, * p<0.05. (D) At day 18, tumors were excised, formalin fixed, and paraffin embedded. Tumor histology was assessed by hemotoxylin and eosin staining A representative image is shown. Magnification: 20×. Scale bar=100 gm. (E) Ramos cells were injected into the s.c. into nude BALB/c mice. As tumors grow rapidly, they were treated systemically twice in the week (Day 0 and 3) with 5mg/kg EMP2 IgG1 or control IgG. Tumor volume values are averages (±SEM, n=6). (F) Representative hematoxylin and eosin staining at day 28. Magnification: 20×. Scale bar =100 μm.

FIG. 5 depicts the reduction of tumor load in orthotopic mammary tumors in the presence of anti-EMP2 antibodies. (A) D2F2 cells are susceptible to apoptosis when treated with anti-EMP2 IgG1 compared to the control IgG. Cells were stained with annexin-V and propidium iodide, and then analyzed by flow cytometry. Images are representative of three independent experiments which showed similar results. (B) D2F2 cells were injected into the mammary pad of BALB/c mice. When tumors were first detectable, mice were systemically injected with 10 mg/kg anti-EMP2 or control IgG twice in the week (Day 0 and 3). Tumor volume was calculated using calipers N=6. *, p<0.05, comparison by Student's t test. (C) Representative images of Day 7 tumors stained with hemotoxlin and eosin. Magnification: 20×. Scale bar, 100 μm.

FIG. 6 depicts the characterization of a recombinant anti-EMP2 IgG1 antibody. An anti-EMP2 IgG1 humanized antibody was constructed. To verify our construct, the anti-EMP2 IgG1 antibody was sequenced and analyzed by SDS-PAGE under non-reducing (NR) and reducing (R) conditions. The 150 KDa band appeared, which exhibited a molecular weight corresponding to our anti-EMP2 IgG1. Anti-EMP2 IgG1 migrated as monomers under reducing condition at an expected 66 kDa size, corresponding to the H-chain (H) and 20 kDa L-chain (L) bands. To characterize the specificity of the anti-EMP2 IgG1, the binding of the full length anti-EMP2 antibody was measured against EMP2 peptide as well as to native protein. Using a human EMP2 peptide, serial dilutions of the native antibody revealed an EC₅₀ of 10.8 ng/ml (B). To further determine the binding characteristics of the anti-EMP2 IgG1, its binding to native EMP2 was determined using flow cytometry. Anti-EMP2 IgG1 recognized both murine EMP2 present on D2F2 cells (C) and human EMP2 on an endometrial carcinoma cell line which overexpresses EMP2, HEC-1a/EMP2 cells (D). The binding of the antibody was specific as it did not bind the EMP2 negative cell line Ramos (E). Finally, the sensitivity of the antibody was measured using HEC-1A wild type and HEC-1A/EMP2 cells which express a two to four-fold increase in total EMP2 levels. Anti-EMP2 IgG1 detected a difference in surface expression between the two cell lines (F).

FIG. 7 depicts the lack of systemic toxicity exhibited by antibodies to EMP2. Anti-EMP2 diabodies (55 kDa) do not exhibit systemic toxicity. The full-length antibody differs from diabody in its valency, molecular size, and presence of immunoglobulin constant region domains which serve to modify the in vivo properties such as tumor uptake and interaction with immune system. To determine if the anti-EMP2 IgG1 produces any histological or sera toxicity in normal BALB/c mice, mice were injected weekly with 10 mg/kg anti-EMP2 IgG1 or sterile saline for seven weeks. Animals treated with either the control of anti-EMP2 IgG1 exhibited no significant differences in weight. Although a slight change in AST and cholesterol was observed, all changes were within the normal range found in mice as provided in Table 1 below. N=4.

TABLE 1

| | Normal Mouse | Treatment | |
|---|---|---|---|
| Serum Protein | Reference | Control IgG | EMP2 IgG1 |
| Albumin (g/dL) | 2.5-5.4 | 3.1 ± 0.2 | 3.0 ± 0.2 |
| Alkaline Phosphatase (U/L) | 13-291 | 55 ± 9.2 | 73.5 ± 6.4 |
| ALT (U/L) | 7-227 | 39.7 ± 3.1 | 49.5 ± 0.7 |
| AST (U/L) | 34-329 | 112.3 ± 20 | 148.0 ± 28.3 |
| Total Bilirubin (mg/dL) | 0.1-1.1 | 0.3 ± 0.1 | 0.3 ± 0.2 |
| Direct Bilirubin (mg/dL) | — | 0.3 ± 0.1 | 0.2 ± 0.1 |
| BUN (mg/dL) | 2.0-5.4 | 16.0 ± 3 | 21.0 ± 5.7 |
| Cholesterol (mg/dL) | 34-219 | 95.7 ± 4.2 | 79.5 ± 2.1 |
| Glucose (mg/dL) | 46-279 | 129.7 ± 27.8 | 135.0 ± 14.1 |
| Total Protein (g/dL) | 3.3-7.6 | 6.3 ± 0.3 | 6.1 ± 0.4 |

FIG. 8 depicts the amino acid sequences of the Heavy and Light chain variable regions of anti-EMP-2 antibodies KS49, KS41, KS89 and KS83 are shown. Suitable CDR sequences of the variable regions are identified using the Kabat CDR definition.

FIG. 9 depicts the amino acid sequences of the Heavy and Light chain variable regions of anti-EMP-2 antibodies KS49, KS41, KS89 and KS83 showing the suitable CDR sequences for use in the antibodies of the invention.

FIG. 10 depicts the amino acid sequences of the KS49, KS41, KS89 and KS83 diabodies with underlining of their linkers and polyhistidine tags.

### DETAILED DESCRIPTION OF THE INVENTION

### Introduction

Breast cancer remains the most common malignancy in women. One particularly aggressive type of breast cancer lacks expression of estrogen-receptor (ER), Her-2/neu and the progesterone receptor (PR). This aggressive type of breast cancer is often referred to as triple negative breast cancer. Triple negative breast cancer accounts for nearly half of all breast cancer deaths. Accordingly, understanding the molecular basis of triple negative breast cancer is necessary to form strategies for detection and treatment.

The Applicants have discovered that EMP2 is highly expressed in over 70% of triple negative breast cancer cases. Methods of diagnosing triple negative breast cancer are known to one of skill in the art. Furthermore, it was previously reported that targeting of EMP2 may offer a therapeutic strategy in treating breast cancer. US Pat. Pub. 20100272732, incorporated by reference in its entirety. Accordingly, in its first aspect, the invention provides compositions of anti-EMP2 antibodies and methods of treating triple negative breast cancer. In a specific aspect, the invention provides the administration of anti-EMP2 antibodies in a physiologically acceptable carrier or a pharmaceutically acceptable carrier. In another aspect, the invention provides compositions of anti-EMP2 antibodies and methods of detecting triple negative breast cancer. In another aspect, the invention provides compositions of anti-EMP2 antibodies and methods of co-administration with one or more additional therapies. In another aspect, the invention provides companion diagnostic methods and products for use with the methods and antibodies described herein.

### Antibodies

Antibodies that find use in the present invention can take on a number of formats such as traditional antibodies as well as antibody derivatives, fragments and mimetics. In certain embodiments of this invention, the anti-EMP2 antibodies are KS49, KS41, KS83, or KS89. These antibodies and their use are described herein.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. It should be understood that therapeutic antibodies can also comprise hybrids of isotypes and/or subclasses.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5^{th} Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. For example, as described herein the antibodies bind to an epitope in the presumptive second extracellular domain of EMP2.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

In some embodiments, the epitope is derived from SEQ ID NO:2, wherein SEQ ID NO:2 is EDIHDKNAKFYPVTREGSYG and represents a 20-mer polypeptide sequence from the second extracellular loop of human EMP2

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5^{th} edition, NIH publication, No. 91-3242, E.A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

Of interest in the present invention are the Fc regions. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cy2 and Cy3 (Cy2 and Cy3) and the lower hinge region between Cy1 (Cy1) and Cy2 (Cy2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Structures that still rely

In one embodiment, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab')2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

In some embodiments, the antibody can be a mixture from different species, e.g. a chimeric antibody and/or a humanized antibody. That is, in the present invention, the CDR sets can be used with framework and constant regions other than those specifically described by sequence herein.

In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; US 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al.,1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O′Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

In one embodiment, the antibodies of the invention can be multispecific antibodies, and notably bispecific antibodies. These are antibodies that bind to two (or more) different antigens, or different epitopes on the same antigen.

In some embodiments the antibodies are diabodies.

In one embodiment, the antibody is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region.

The antibodies of the present invention are generally isolated or recombinant. An “isolated antibody,” refers to an antibody which is substantially free of other antibodies having different antigenic specificities. For instance, an isolated antibody that specifically binds to EMP2 is substantially free of antibodies that specifically bind antigens other than EMP2.

An isolated antibody that specifically binds to an epitope, isoform or variant of human EMP2 or murine EMP2 may, however, have cross-reactivity to other related antigens, for instance from other species, such as EMP2 species homologs. Moreover, an isolated antibody may be substantially free of other cellular material and/or chemicals.

Isolated monoclonal antibodies, having different specificities, can be combined in a well defined composition. Thus, for example all possible combinations of the antibodies KS49, KS41, KS83, or KS89 can be combined in a single formulation, if desired.

The following human-origin antibody sequences encode for high-avidity antibodies specific for human (KS49, KS83) and mouse (KS83) EMP2 and have antibody variable region heavy and light chains suitable for use in either aspect of the invention:

KS49 heavy chain-
M A Q V Q L V Q S G G G V V Q P G R S L R L S
C A A S G F T F S S Y A M H W V R Q A P G K G
L E W V A V I S Y D G S N K Y Y A D S V K G R
F T I S R D N S K N T L Y L Q M N S L R A E D
T A V Y Y C A R D R R G R K S A G I D Y W G Q
G T L V T V S S
KS49 light chain-
D I Q M T Q S P S S L S A S V G D R V T I T C
Q A S Q D I S N Y L N W Y Q Q K P G K A P K L
L I Y A A S S L Q S G V P S R F S G S G S G T
D F T L T I S S L Q P E D F A T Y Y C L Q D Y
N G W T F G Q G T K V D I K R A A A E Q K L I
S E E D L N G A A
KS83 heavy chain-
M A Q V Q L V E S G G G L V Q P G G S L R L S
C A A S G F T F S S Y A M H W V R Q A P G K G
L E W V A V I S Y D G S N K Y Y A D S V K G R
F T I S R D N S K N T L Y L Q M N S L R A E D
T A V Y Y C A R T V G A T G A F D I W G Q G T
M V T V S S S
KS83 light chain-
D I V M T Q S P S T V S A S V G D R V I I P C
R A S Q S I G K W L A W Y Q Q K P G K A P K L
L I Y K A S S L E G W V P S R F S G S G S G T
E F S L T I S S L Q P D D S A T Y V C Q Q S H
N F P P T F G G G T K L E I K R A A A E Q K L
I S E E D L N G A A

Other diabodies for use according to either aspect of the invention include KS41 and KS89:

KS41 Heavy Chain-
M A Q V Q L V Q S G G G L V Q P G R S L R L S
C A A S G F S F S E Y P M H W V R Q A P G R G
L E S V A V I S Y D G E Y Q K Y A D S V K G R
F T I S R D D S K S T V Y L Q M N S L R P E D
T A V Y Y C A R T I N N G M D V W G Q G T T V
T V S S
KS41 Light Chain-
D I V M T Q S P S S L S A S V G D R V T I T C
R A S Q G I R N D L G W Y Q Q K P G K A P E L
L I Y G A S S L Q S G V P S R F S G S G S G T
D F T L T I S S L Q P E D S A T Y Y C L Q D Y
N G W T F G Q G T K L E I K R A A A E Q K L I
S E E D L N G A A
KS89 Heavy Chain-
M A Q V Q L V Q S G G G L V Q P G R S L R L S
C A A S G F S F S E Y P M H W V R Q A P G R G
L E S V A V I S Y D G E Y Q K Y A D S V K G R
F T I S R D D S K S T V Y L Q M N S L R P E D
T A V Y Y C A R T I N N G M D V W G Q G T T V
T V S S
KS89 Light Chain-
D I V M T Q S P S S L S A S V G D R V T I T C
R A S Q G I R N D L G W Y Q Q K P G K A P E L
L I Y G A S S L Q S G V P S R F S G S G S G T
D F T L T I S S L Q P E D S A T Y Y C L Q D Y
N G W T F G Q G T K L E I K R A A A E Q K L I
S E E D L N G A A

Anti-EMP-2 variable region sequences, used to encode proteins on backbones including for native antibody, fragment antibody, or synthetic backbones, can avidly bind EMP-2. Via this binding, these proteins can be used for EMP-2 detection, and to block EMP-2 function. Expression of these variable region sequences on native antibody backbones, or as an scFv, triabody, diabody or minibody, labeled with radionuclide, are particularly useful in in the in vivo detection of EMP-2 bearing cells. Expression on these backbones or native antibody backbone are favorable for blocking the function of EMP-2 and/or killing EMP-2 bearing cells (e.g.gynecologic tumors) in vivo.

In some embodiments, the present invention provides anti-EMP-2 sequences comprising CDR regions of an antibody selected from KS49, KS83, KS41, and KS89, as shown in FIG. 8. The CDR regions provided by the invention may be used to construct an anti-EMP-2 binding protein, including without limitation, an antibody, a scFv, a triabody, a diabody, a minibody, and the like. In a certain embodiment, an anti-EMP-2 binding protein of the invention will comprise at least one CDR region from an antibody selected from KS49, KS83, KS41, and KS89. Anti-EMP-2 binding proteins may comprise, for example, a CDR-H1, a CDR-H2, a CDR-H3, a CDR-L1, a CDR-L2, a CDR-L3, or combinations thereof, from an antibody provided herein. In particular embodiments of the invention, an anti-EMP-2 binding protein may comprise all three CDR-H sequences of an antibody provided herein, all three CDR-L sequences of an antibody provided herein, or both. Anti-EMP2 CDR sequences may be used on an antibody backbone, or fragment thereof, and likewise may include humanized antibodies, or antibodies containing humanized sequences. These antibodies may be used, for example, to detect EMP-2, to detect cells expressing EMP-2 in vivo, or to block EMP-2 function. In some embodiments, the CDR regions may be defined using the Kabat definition, the Chothia definition, the AbM definition, the contact definition, or any other suitable CDR numbering system.

In some embodiments, the CDRs are as follows:

CDR 1 Heavy
SYAMH (49)
SYAMH (83)
EYPMH (41)
EYPMH (89)
CDR 2 Heavy
VISYDGSNKYYADSVKG (49)
VISYDGSNKYYADSVKG (83)
VISYDGEYQKYADSVKG (41)
VISYDGEYQKYADSVKG (89)
CDR 1 Light
QASQDISNYLN (49)
RASQSIGKWLA (83)
RASQGIRNDLG (41)
RASQGIRNDLG (89)
CDR 2 Light
AASSLQS (49)
KASSLEG (83)
GASSLQS (41)
GASSLQS (89)
Diabody sequence (KS49)
Heavy chain, KS49
M A Q V Q L V Q S G G G V V Q P G R S L R L S C A
A S G F T F S S Y A M H W V R Q A P G K G L E W V
A V I S Y D G S N K Y Y A D S V K G R F T I S R D
N S K N T L Y L Q M N S L R A E D T A V Y Y C A R
D R R G R K S A G I D Y W G Q G T L V T V S
CDR1 SYAMH
CDR2 VISYDGSNKYYADSVKG
Light chain, KS49
D I Q M T Q S P S S L S A S V G D R V T I T C Q A
S Q D I S N Y L N W Y Q Q K P G K A P K L L I Y A
A S S L Q S G V P S R F S G S G S G T D F T L T I
S S L Q P E D F A T Y Y C L Q D Y N G W T F G Q G
T K V D I K R A A A E Q K L I S E E D L N G A A
CDR 1 QASQDISNYLN
CDR2 AASSLQS
Diabody sequence (KS83)
Heavy chain, KS83
M A Q V Q L V E S G G G L V Q P G G S L R L S C A
A S G F T F S S Y A M H W V R Q A P G K G L E W V
A V I S Y D G S N K Y Y A D S V K G R F T I S R D
N S K N T L Y L Q M N S L R A E D T A V Y Y C A R
T V G A T G A F D I W G Q G T M V T V S S
CDR1 SYAMH
CDR2 VISYDGSNKYYADSVKG
Light Chain, KS83
D I V M T Q S P S T V S A S V G D R V I I P C R A
S Q S I G K W L A W Y Q Q K P G K A P K L L I Y K
A S S L E G W V P S R F S G S G S G T E F S L T I
S S L Q P D D S A T Y V C Q Q S H N F P P T F G G
G T K L E I K R A A A E Q K L I S E E D L N G A A
CDR1 RASQSIGKWLA
CDR2 KASSLEG
Diabody sequence (KS41)
Heavy Chain, KS41
M A Q V Q L V Q S G G G L V Q P G R S L R L S C A
A S G F S F S E Y P M H W V R Q A P G R G L E S V
A V I S Y D G E Y Q K Y A D S V K G R F T I S R D
D S K S T V Y L Q M N S L R P E D T A V Y Y C A R
T I N N G M D V W G Q G T T V T V S S
CDR 1 EYPMH
CDR 2 VISYDGEYQKYADSVKG
Light Chain, KS41
D I V M T Q S P S S L S A S V G D R V T I T C R A
S Q G I R N D L G W Y Q Q K P G K A P E L L I Y G
A S S L Q S G V P S R F S G S G S G T D F T L T I
S S L Q P E D S A T Y Y C L Q D Y N G W T F G Q G
T K L E I K R A A A E Q K L I S E E D L N G A A
CDR 1 RASQGIRNDLG
CDR 2 GASSLQS
Diabody sequence (KS89)
Heavy Chain, KS89
M A Q V Q L V Q S G G G L V Q P G R S L R L S C A
A S G F S F S E Y P Mt H W V R Q A P G R G L E S V
A V I S Y D G E Y Q K Y A D S V K G R F T I S R D
D D S K S T V Y L Q M N S L R P E D T A V Y Y C A
R T I N N G M D V W G Q G T T V T V S S
CDR1 EYPMH
CDR 2 VISYDGEYQKYADSVKG
Light Chain, KS89
D I V Met T Q S P S S L S A S V G D R V T I T C R
A S Q G I R N D L G W Y Q Q K P G K A P E L L I Y
G A S S L Q S G V P S R F S G S G S G T D F T L T
I S S L Q P E D S A T Y Y C L Q D Y N G W T F G Q
G T K L E I K R A A A E Q K L I S E E D L N G A A
CDR 1 RASQGIRNDLG
CDR 2 GASSLQS

In some embodiments, the invention provides antibodies (e.g., diabodies, minibodies, triabodies) or fragments thereof having the CDRs of a diabody selected from KS49, KS83, KS41, and KS89. In some embodiments these antibodies lack the polyhistine tag. In other embodiments, the diabodies possess the light and heavy chain of a KS49, KS83, KS41, or KS89 diabody. In stillother embodiments, the antibodies are substantially identical in sequence to a diabody selected from the group consisting of KS49, KS83, KS41, and KS89 with or without the polyhistidine tag. In stillother embodiments, the antibodies are substantially identical in sequence to the light and heavy chain sequences of a diabody selected from the group consisting of KS49, KS83, KS41, and KS89. These identities can be 65%, 70%, 75%, 80%, 85%, 90%, and preferably 91%, 92%, 93%, 94%, 95%, 96%, 97%, 98% or 99% or greater amino acid sequence identity. In some further embodiments of any of the above, the antibodies comprise CDRs sequences identical to those of the KS49, KS83, KS41, or KS89 diabody.

The anti-EMP2 antibodies of the present invention specifically bind EMP2 ligands (e.g. the human and murine EMP2 proteins of SEQ ID NOs:1 and 2.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The present invention further provides variant antibodies. That is, there are a number of modifications that can be made to the antibodies of the invention, including, but not limited to, amino acid modifications in the CDRs (affinity maturation), amino acid modifications in the Fc region, glycosylation variants, covalent modifications of other types, etc.

By “variant” herein is meant a polypeptide sequence that differs from that of a parent polypeptide by virtue of at least one amino acid modification. Amino acid modifications can include substitutions, insertions and deletions, with the former being preferred in many cases.

In general, variants can include any number of modifications, as long as the function of the protein is still present, as described herein. That is, in the case of amino acid variants generated with the CDRs of KS49, KS41, KS83, or KS89, for example, the antibody should still specifically bind to both human and/or murine EMP2. Similarly, if amino acid variants are generated with the Fc region, for example, the variant antibodies should maintain the required receptor binding functions for the particular application or indication of the antibody.

However, in general, from 1, 2, 3, 4, 5, 6, 7, 8, 9 or 10 amino acid substitutions are generally utilized as often the goal is to alter function with a minimal number of modifications. In some cases, there are from 1 to 5 modifications, with from 1-2, 1-3 and 1-4 also finding use in many embodiments.

It should be noted that the number of amino acid modifications may be within functional domains: for example, it may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 95% or up to 98 or 99% identity to the parent sequences (e.g. the variable regions, the constant regions, and/or the heavy and light chain sequences for KS49, KS41, KS83, or KS89. It should be noted that depending on the size of the sequence, the percent identity will depend on the number of amino acids.

By “amino acid substitution” or “substitution” herein is meant the replacement of an amino acid at a particular position in a parent polypeptide sequence with another amino acid. For example, the substitution S100A refers to a variant polypeptide in which the serine at position 100 is replaced with alanine By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

By “parent polypeptide”, “parent protein”, “precursor polypeptide”, or “precursor protein” as used herein is meant an unmodified polypeptide that is subsequently modified to generate a variant. In general, the parent polypeptides herein are Ab79 and Ab19. Parent polypeptide may refer to the polypeptide itself, compositions that comprise the parent polypeptide, or the amino acid sequence that encodes it. Accordingly, by “parent Fc polypeptide” as used herein is meant an Fc polypeptide that is modified to generate a variant, and by “parent antibody” as used herein is meant an antibody that is modified to generate a variant antibody.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

By “variant Fc region” herein is meant an Fc sequence that differs from that of a wild-type Fc sequence by virtue of at least one amino acid modification. Fc variant may refer to the Fc polypeptide itself, compositions comprising the Fc variant polypeptide, or the amino acid sequence.

In some embodiments, one or more amino acid modifications are made in one or more of the CDRs of the antibody (KS49, KS41, KS83, or KS89). In general, only 1 or 2 or 3amino acids are substituted in any single CDR, and generally no more than from 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

Affinity maturation can be done to increase the binding affinity of the antibody for the antigen by at least about 10% to 50-100-150% or more, or from 1 to 5 fold as compared to the “parent” antibody. Preferred affinity matured antibodies will have nanomolar or even picomolar affinities for the target antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of KS49, KS41, KS83, or KS89. In addition, as outlined below, amino acid modifications can also independently and optionally be made in any region outside the CDRs, including framework and constant regions.

In some embodiments, the anti-EMP2 antibodies of the invention are composed of a variant Fc domain. As is known in the art, the Fc region of an antibody interacts with a number of Fc receptors and ligands, imparting an array of important functional capabilities referred to as effector functions. These Fc receptors include, but are not limited to, (in humans) FcγRI (CD64) including isoforms FcγRIa, FcγRIb, and FcγRIc; FcyRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158, correlated to antibody-dependent cell cytotoxicity (ADCC)) and FcγRIIIb (including allotypes FcγRIIIb-NA1 and FcγRIIIb-NA2), FcRn (the neonatal receptor), Clq (complement protein involved in complement dependent cytotoxicity (CDC)) and FcRn (the neonatal receptor involved in serum half-life). Suitable modifications can be made at one or more positions as is generally outlined, for example in

U.S. patent application Ser. No. 11/841,654 and references cited therein, US 2004/013210, US 2005/0054832, US 2006/0024298, US 2006/0121032, US 2006/0235208, US 2007/0148170, U.S. Ser. No. 12/341,769, U.S. Pat. No. 6,737,056, U.S. Pat. No. 7,670,600, U.S. Pat. No. 6,086,875 all of which are expressly incorporated by reference in their entirety, and in particular for specific amino acid substitutions that increase binding to Fc receptors.

In addition to the modifications outlined above, other modifications can be made. For example, the molecules may be stabilized by the incorporation of disulphide bridges linking the VH and VL domains (Reiter et al., 1996, Nature Biotech. 14:1239-1245, entirely incorporated by reference). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues may also be derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole and the like.

In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cynomolgusogen bromide-activated carbohydrates and the reactive substrates described in U.S. Pat. Nos. 3,969,287; 3,691,016; 4,195,128; 4,247,642; 4,229,537; and 4,330,440, all entirely incorporated by reference, are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In addition, as will be appreciated by those in the art, labels (including fluorescent, enzymatic, magnetic, radioactive, etc. can all be added to the antibodies (as well as the other compositions of the invention).

Another type of covalent modification is alterations in glycosylation. In another embodiment, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. A preferred form of engineered glycoform is afucosylation, which has been shown to be correlated to an increase in ADCC function, presumably through tighter binding to the FcγRIIIa receptor. In this context, “afucosylation” means that the majority of the antibody produced in the host cells is substantially devoid of fucose, e.g. 90-95-98% of the generated antibodies do not have appreciable fucose as a component of the carbohydrate moiety of the antibody (generally attached at N297 in the Fc region). Defined functionally, afucosylated antibodies generally exhibit at least a 50% or higher affinity to the FcγRIIIa receptor.

Engineered glycoforms may be generated by a variety of methods known in the art (Umaria et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zürich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells, by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. For example, the “sugar engineered antibody” or “SEA technology” of Seattle Genetics functions by adding modified saccharides that inhibit fucosylation during production; see for example 20090317869, hereby incorporated by reference in its entirety. Engineered glycoform typically refers to the different carbohydrate or oligosaccharide; thus an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin and Wriston, 1981, CRC Crit. Rev. Biochem., pp. 259-306, both entirely incorporated by reference.

Removal of carbohydrate moieties present on the starting antibody (e.g. post-translationally) may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference.

The present invention provides a number of antibodies each with a specific set of CDRs (including, as outlined above, some amino acid substitutions). As outlined above, the antibodies can be defined by sets of 6 CDRs, by variable regions, or by full-length heavy and light chains, including the constant regions. In addition, as outlined above, amino acid substitutions may also be made. In general, in the context of changes within CDRs, due to the relatively short length of the CDRs, the amino acid modifications are generally described in terms of the number of amino acid modifications that may be made. While this is also applicable to the discussion of the number of amino acid modifications that can be introduced in variable, constant or full length sequences, in addition to number of changes, it is also appropriate to define these changes in terms of the “% identity”. Thus, as described herein, antibodies included within the invention are 80, 85, 90, 95, 98 or 99% identical to KS49, KS41, KS83, or KS89 described herein.

In some embodiments, antibodies that compete with the antibodies of the invention (for example, with KS49, KS41, KS83, or KS89) for binding to human EMP2 and/or murine EMP2 are provided. Competition for binding to EMP2 or a portion of EMP2 by two or more anti-EMP2 antibodies may be determined by any suitable technique, as is known in the art.

Competition in the context of the present invention refers to any detectably significant reduction in the propensity of an antibody of the invention (e.g. KS49, KS41, KS83, or KS89) to bind its particular binding partner, e.g. EMP2, in the presence of the test compound. Typically, competition means an at least about 10-100% reduction in the binding of an antibody of the invention to EMP2 in the presence of the competitor, as measured by standard techniques such as ELISA or Biacore® assays. Thus, for example, it is possible to set criteria for competitiveness wherein at least about 10% relative inhibition is detected; at least about 15% relative inhibition is detected; or at least about 20% relative inhibition is detected before an antibody is considered sufficiently competitive. In cases where epitopes belonging to competing antibodies are closely located in an antigen, competition may be marked by greater than about 40% relative inhibition of EMP2 binding (e.g., at least about 45% inhibition, such as at least about 50% inhibition, for instance at least about 55% inhibition, such as at least about 60% inhibition, for instance at least about 65% inhibition, such as at least about 70% inhibition, for instance at least about 75% inhibition, such as at least about 80% inhibition, for instance at least about 85% inhibition, such as at least about 90% inhibition, for instance at least about 95% inhibition, or higher level of relative inhibition).

In some cases, one or more of the components of the competitive binding assays are labeled.

It may also be the case that competition may exist between anti-EMP2 antibodies with respect to more than one of EMP2 epitope, and/or a portion of EMP2, e.g. in a context where the antibody-binding properties of a particular region of EMP2 are retained in fragments thereof, such as in the case of a well-presented linear epitope located in various tested fragments or a conformational epitope that is presented in sufficiently large EMP2 fragments as well as in EMP2.

Assessing competition typically involves an evaluation of relative inhibitory binding using an antibody of the invention, EMP2 (either human or murine or both), and the test molecule. Test molecules can include any molecule, including other antibodies, small molecules, peptides, etc. The compounds are mixed in amounts that are sufficient to make a comparison that imparts information about the selectivity and/or specificity of the molecules at issue with respect to the other present molecules.

The amounts of test compound, EMP2 and antibodies of the invention may be varied. For instance, for ELISA assessments about 5-50 μg (e.g., about 10-50 μg, about 20-50 μg, about 5-20 μg, about 10-20 μg, etc.) of the anti-EMP2 antibody and/or EMP2 targets are required to assess whether competition exists. Conditions also should be suitable for binding. Typically, physiological or near-physiological conditions (e.g., temperatures of about 20-40° C., pH of about 7-8, etc.) are suitable for anti-EMP2:EMP2 binding.

Often competition is marked by a significantly greater relative inhibition than about 5% as determined by ELISA and/or FACS analysis. It may be desirable to set a higher threshold of relative inhibition as a criteria/determinant of what is a suitable level of competition in a particular context (e.g., where the competition analysis is used to select or screen for new antibodies designed with the intended function of blocking the binding of another peptide or molecule binding to EMP2 (e.g., the natural binding partners of EMP2 or naturally occurring anti-EMP2 antibody).

In some embodiments, the anti-EMP2 antibody of the present invention specifically binds to one or more residues or regions in EMP2 but also does not cross-react with other proteins with homology to EMP2.

Typically, a lack of cross-reactivity means less than about 5% relative competitive inhibition between the molecules when assessed by ELISA and/or FACS analysis using sufficient amounts of the molecules under suitable assay conditions.

The disclosed antibodies may find use in blocking a ligand-receptor interaction or inhibiting receptor component interaction. The anti-EMP2 antibodies of the invention may be “blocking” or “neutralizing.” A “neutralizing antibody” is intended to refer to an antibody whose binding to EMP2 results in inhibition of the biological activity of EMP2, for example its capacity to interact with ligands, enzymatic activity, and/or signaling capacity Inhibition of the biological activity of EMP2 can be assessed by one or more of several standard in vitro or in vivo assays known in the art.

Inhibits binding” or “blocks binding” (for instance when referring to inhibition/blocking of binding of a EMP2 binding partner to EMP2) encompass both partial and complete inhibition/blocking The inhibition/blocking of binding of a EMP2 binding partner to EMP2 may reduce or alter the normal level or type of cell signaling that occurs when a EMP2 binding partner binds to EMP2 without inhibition or blocking Inhibition and blocking are also intended to include any measurable decrease in the binding affinity of a EMP2 binding partner to EMP2 when in contact with an anti-EMP2 antibody, as compared to the ligand not in contact with an anti-EMP2 antibody, for instance a blocking of binding of a EMP2 binding partner to EMP2 by at least about 10%, 20%, 30%, 40%, 50%, 60%, 70%, 80%, 90%, 99%, or 100%.

The present invention further provides methods for producing the disclosed anti-EMP2 antibodies. These methods encompass culturing a host cell containing isolated nucleic acid(s) encoding the antibodies of the invention. As will be appreciated by those in the art, this can be done in a variety of ways, depending on the nature of the antibody. In some embodiments, in the case where the antibodies of the invention are full length traditional antibodies, for example, a heavy chain variable region and a light chain variable region under conditions such that an antibody is produced and can be isolated.

In general, nucleic acids are provided that encode the antibodies of the invention. Such polynucleotides encode for both the variable and constant regions of each of the heavy and light chains, although other combinations are also contemplated by the present invention in accordance with the compositions described herein. The present invention also contemplates oligonucleotide fragments derived from the disclosed polynucleotides and nucleic acid sequences complementary to these polynucleotides.

The polynucleotides can be in the form of RNA or DNA. Polynucleotides in the form of DNA, cDNA, genomic DNA, nucleic acid analogs, and synthetic DNA are within the scope of the present invention. The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

In some embodiments, nucleic acid(s) encoding the antibodies of the invention are incorporated into expression vectors, which can be extrachromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, etc.) or other components (selection genes, etc.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, etc.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, etc.), whereby the encoded polypeptide(s) are produced. In some cases, the heavy chains are produced in one cell and the light chain in another.

Mammalian cell lines available as hosts for expression are known in the art and include many immortalized cell lines available from the American Type Culture Collection (ATCC), Manassas, Vir. including but not limited to Chinese hamster ovary (CHO) cells, HEK 293 cells, NSO cells, HeLa cells, baby hamster kidney (BHK) cells, monkey kidney cells (COS), human hepatocellular carcinoma cells (e.g., Hep G2), and a number of other cell lines. Non-mammalian cells including but not limited to bacterial, yeast, insect, and plants can also be used to express recombinant antibodies. In some embodiments, the antibodies can be produced in transgenic animals such as cows or chickens.

### Methods of Treatment

Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to provide antibodies with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Administrative Modalities

The antibodies and chemotherapeutic agents of the invention are administered to a subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In certain aspects, the antibodies and chemotherapeutic agents of the invention are administered to a subject with cancer. In certain aspects, the antibodies and chemotherapeutic agents of the invention are administered to a subject with breast cancer. In certain aspects, the antibodies and chemotherapeutic agents of the invention are administered to a subject with triple negative breast cancer. Intravenous or subcutaneous administration of the antibody is preferred.

Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling.

In addition to these positive therapeutic responses, the subject undergoing therapy may experience the beneficial effect of an improvement in the symptoms associated with the disease.

Such a response may persist for at least 4 to 8 weeks, or sometimes 6 to 8 weeks, following treatment according to the methods of the invention. Alternatively, an improvement in the disease may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the anti-EMP2 antibodies used in the present invention depend on the disease or condition to be treated and may be determined by the persons skilled in the art.

An exemplary, non-limiting range for a therapeutically effective amount of an anti-EMP2 antibody used in the present invention is about 0.1-100 mg/kg, such as about 0.1-50 mg/kg, for example about 0.1-20 mg/kg, such as about 0.1-10 mg/kg, for instance about 0.5, about such as 0.3, about 1, or about 3 mg/kg. In another embodiment, he antibody is administered in a dose of 1 mg/kg or more, such as a dose of from 1 to 20 mg/kg, e.g. a dose of from 5 to 20 mg/kg, e.g. a dose of 8 mg/kg.

A medical professional having ordinary skill in the art may readily determine and prescribe the effective amount of the pharmaceutical composition required. For example, a physician or a veterinarian could start doses of the medicament employed in the pharmaceutical composition at levels lower than that required in order to achieve the desired therapeutic effect and gradually increase the dosage until the desired effect is achieved.

In one embodiment, the anti-EMP2 antibody is administered by infusion in a weekly dosage of from 10 to 500 mg/kg such as from 200 to 400 mg/kg. Such administration may be repeated, e.g., 1 to 8 times, such as 3 to 5 times. The administration may be performed by continuous infusion over a period of from 2 to 24 hours, such as from 2 to 12 hours.

In one embodiment, the anti-EMP2 antibody is administered by slow continuous infusion over a long period, such as more than 24 hours, if required to reduce side effects including toxicity.

In one embodiment the anti-EMP2 antibody is administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as from 2 to 12 hours. Such regimen may be repeated one or more times as necessary, for example, after 6 months or 12 months. The dosage may be determined or adjusted by measuring the amount of compound of the present invention in the blood upon administration by for instance taking out a biological sample and using anti-idiotypic antibodies which target the antigen binding region of the anti-EMP2 antibody.

In a further embodiment, the anti-EMP2 antibody is administered once weekly for 2 to 12 weeks, such as for 3 to 10 weeks, such as for 4 to 8 weeks.

In one embodiment, the anti-EMP2 antibody is administered by maintenance therapy, such as, e.g., once a week for a period of 6 months or more.

In one embodiment, the anti-EMP2 antibody is administered by a regimen including one infusion of an anti-EMP2 antibody followed by an infusion of an anti-EMP2 antibody conjugated to a radioisotope. The regimen may be repeated, e.g., 7 to 9 days later.

As non-limiting examples, treatment according to the present invention may be provided as a daily dosage of an antibody in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

Combination Therapy

In some embodiments the anti-EMP2 antibody molecule thereof is used in combination with one or more additional therapeutic agents, e.g. a chemotherapeutic agent.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and other inhibitors of proteins or enzymes known to be upregulated, over-expressed or activated in cancers, the inhibition of which downregulates cell replication.

In some embodiments, the antibodies of the invention can be used prior to, concurrent with, or after treatment with any of the chemotherapeutic agents described herein or known to the skilled artisan at this time or subsequently.

### Efficacy of Methods Described Herein

In certain aspects of this invention, efficacy of anti-EMP2 therapy is measured by decreased serum concentrations of tumor specific markers, increased overall survival time, decreased tumor size, cancer remission, decreased metastasis marker response, and decreased chemotherapy adverse affects.

In certain aspects of this invention, efficacy is measured with companion diagnostic methods and products. Companion diagnostic measurements can be made before, during, or after anti-EMP2 treatment.

### Companion Diagnostics

In other embodiments, this disclosure relates to companion diagnostic methods and products. In one embodiment, the companion diagnostic method and products can be used to monitor the treatment of breast cancer, specifically triple negative breast cancer, as described herein. In some embodiments, the companion diagnostic methods and products include molecular assays to measure levels of proteins, genes or specific genetic mutations. Such measurements can be used, for example, to predict whether anti-EMP2 therapy will benefit a specific individual, to predict the effective dosage of anti-EMP2 therapy, to monitor anti-EMP2 therapy, adjust anti-EMP2 therapy, tailor the anti-EMP2 therapy to an individual, and track cancer progression and remission.

In some embodiments, the companion diagnostic can be used to monitor a combination therapy.

In some embodiments, the companion diagnostic can include an anti-EMP2 antibody described herein.

In some embodiments, the companion diagnostic can be used before, during, or after anti-EMP2 therapy.

### Articles of Manufacture

In other embodiments, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The following references are incorporated by reference in their entirety.
1. Elias A D. Triple negative breast cancer: a short review. Am J Clin Oncol. Dec 2010;33(6):637-645.
2. Foulkes W D, Smith I E, Reis-Filho J S. Triple negative breast cancer. N Engl J Med. Nov. 11 2010;363(20):1938-1948.
3. Weigelt B, Peterse J L, van 't Veer L J. Breast cancer metastasis: markers and models. Nat Rev Cancer. August 2005;5(8):591-602.
4. Paik S, Kim C, Wolmark N. HER2 status and benefit from adjuvant trastuzumab in breast cancer. N Engl J Med. Mar 27 2008;358(13):1409-1411.
5. Al Saleh S, Sharaf L H, Luqmani Y A. Signalling pathways involved in endocrine resistance in breast cancer and associations with epithelial to mesenchymal transition (Review). Int J Oncol. May 2011;38(5):1197-1217.
6. Al-Ejeh F, Smart C E, Morrison B J, et al. Breast cancer stem cells: treatment resistance and therapeutic opportunities. Carcinogenesis. May 2011;32(5):650-658.
7. Rivera E, Gomez H. Chemotherapy resistance in metastatic breast cancer: the evolving role of ixabepilone. Breast Cancer Res. 2010;12 Suppl 2:S2.
8. Osborne C K, Schiff R. Mechanisms of endocrine resistance in breast cancer. Annu Rev Med. Feb 18 2011;62:233-247.
9. Pal S K, Childs B H, Pegram M. Triple negative breast cancer: unmet medical needs. Breast Cancer Res Treat. February 2011;125(3):627-636.
10. Fu M, Maresh E L, Soslow R A, et al. Epithelial membrane protein-2 is a novel therapeutic target in ovarian cancer. Clin Cancer Res. Aug. 1 2010;16(15):3954-3963.
11. Habeeb O, Goodglick L, Soslow R A, et al. Epithelial membrane protein-2 expression is an early predictor of endometrial cancer development. Cancer. Oct. 15 2010;116(20):4718-4726.
12. Wadehra M, Natarajan S, Seligson D B, et al. Expression of epithelial membrane protein-2 is associated with endometrial adenocarcinoma of unfavorable outcome. Cancer. Jul. 1 2006;107(1):90-98.
13. Morales S A, Mareninov S, Coulam P, et al. Functional consequences of interactions between FAK and epithelial membrane protein 2 (EMP2). Invest Ophthalmol Vis Sci. October 2009;50(10):4949-4956.
14. Morales S A, Mareninov S, Prasad P, Wadehra M, Braun J, Gordon L K. Collagen gel contraction by ARPE-19 cells is mediated by a FAK-Src dependent pathway. Exp Eye Res. Dec 2007;85(6):790-798.
15. Morales S A, Mareninov S, Wadehra M, et al. FAK activation and the role of epithelial membrane protein 2 (EMP2) in collagen gel contraction. Invest Ophthalmol Vis Sci. January 2009;50(1):462-469.
16. Wadehra M, Forbes A, Pushkarna N, et al. Epithelial membrane protein-2 regulates surface expression of alphavbeta3 integrin in the endometrium. Dev Biol. Nov. 15 2005;287(2):336-345.
17. Hakomori S I. Glycosynaptic microdomains controlling tumor cell phenotype through alteration of cell growth, adhesion, and motility. FEBS Lett. May 3 2010;584(9):1901-1906.
18. Shimazaki K, Lepin E J, Wei B, et al. Diabodies targeting epithelial membrane protein 2 reduce tumorigenicity of human endometrial cancer cell lines. Clin Cancer Res. Nov. 15 2008;14(22):7367-7377.
19. van 't Veer L J, Dai H, van de Vijver M J, et al. Gene expression profiling predicts clinical outcome of breast cancer. Nature. Jan. 31 2002;415(6871):530-536.
20. Obermayr E, Sanchez-Cabo F, Tea M K, et al. Assessment of a six gene panel for the molecular detection of circulating tumor cells in the blood of female cancer patients. BMC Cancer. 2010;10:666.
21. Kao J, Salari K, Bocanegra M, et al. Molecular profiling of breast cancer cell lines defines relevant tumor models and provides a resource for cancer gene discovery. PLoS One. 2009;4(7):e6146.
22. Shimazaki K, Wadehra M, Forbes A, et al. Epithelial membrane protein 2 modulates infectivity of Chlamydia muridarum (MoPn). Microbes Infect. July 2007;9(8):1003-1010.
23. Wei W Z, Shi WP, Galy A, et al. Protection against mammary tumor growth by vaccination with full-length, modified human ErbB-2 DNA. Int J Cancer. May 31 1999;81(5):748-754.
24. Habeeb O, Goodglick L, Soslow R A, et al. Epithelial membrane protein-2 expression is an early predictor of endometrial cancer development. Cancer. Jun. 24 2010.
25. Shimazaki K, Chan A M, Moniz R J, et al. Blockade of epithelial membrane protein 2 (EMP2) abrogates infection of Chlamydia muridarum murine genital infection model. FEMS Immunol Med Microbiol. March 2009;55(2):240-249.
26. Xuan C, Steward K K, Timmerman J M, Morrison S L. Targeted delivery of interferon-alpha via fusion to anti-CD20 results in potent antitumor activity against B-cell lymphoma. Blood. Apr. 8 2010;115(14):2864-2871.
27. Helguera G, Penichet M L. Antibody-cytokine fusion proteins for the therapy of cancer. Methods Mol Med. 2005;109:347-374.
28. Qu Z, Griffiths G L, Wegener W A, et al. Development of humanized antibodies as cancer therapeutics. Methods. 2005;36(1):84-95.
29. Morrison S L, Wims L, Wallick S, Tan L, Oi V T. Genetically engineered antibody molecules and their application. Ann N Y Acad Sci. 1987;507:187-198.
30. Yoon N K, Maresh E L, Elshimali Y, et al. Elevated MED28 expression predicts poor outcome in women with breast cancer. BMC Cancer. 2010;10:335.
31. Yoon N K, Maresh E L, Shen D, et al. Higher levels of GATA3 predict better survival in women with breast cancer. Hum Pathol. December 2010;41(12):1794-1801.
32. Yoon N K, Seligson D B, Chia D, et al. Higher expression levels of 14-3-3sigma in ductal carcinoma in situ of the breast predict poorer outcome. Cancer Biomark. 2009;5(4):215-224.
33. Shen D, Nooraie F, Elshimali Y, et al. Decreased expression of annexin Al is correlated with breast cancer development and progression as determined by a tissue microarray analysis. Hum Pathol. December 2006;37(12):1583-1591.
34. Mah V, Seligson D B, Li A, et al. Aromatase expression predicts survival in women with early-stage non small cell lung cancer. Cancer Res. Nov. 1 2007;67(21):10484-10490.
35. Presson A P, Yoon N K, Bagryanova L, et al. Protein expression based multimarker analysis of breast cancer samples. BMC Cancer. 2011;11:230.
36. Rakha E A, El-Sayed M E, Green A R, Lee A H, Robertson J F, Ellis I O. Prognostic markers in triple negative breast cancer. Cancer. Jan 1 2007;109(1):25-32.
37. Subik K, Lee J F, Baxter L, et al. The Expression Patterns of ER, PR, HER2, CK5/6, EGFR, Ki-67 and AR by Immunohistochemical Analysis in Breast Cancer Cell Lines. Breast Cancer (Auckl). 2010;4:35-41.
38. Holliger P, Prospero T, Winter G. “Diabodies”: small bivalent and bispecific antibody fragments. Proc Natl Acad Sci USA. Jul. 15 1993;90(14):6444-6448.
39. Fu M, Rao R, Sudhakar D, et al. Epithelial Membrane Protein-2 Promotes Endometrial Tumor Formation through Activation of FAK and Src. PLoS One. 2011;6(5):e19945.
40. Haffty B G, Yang Q, Reiss M, et al. Locoregional relapse and distant metastasis in conservatively managed triple negative early-stage breast cancer. J Clin Oncol. Dec 20 2006;24(36):5652-5657.
41. Wadehra M, Forbes A, Pushkarna N, et al. Epithelial membrane protein-2 regulates surface expression of alphavbeta3 integrin in the endometrium. DevBiol. 2005;287:336-345.
42. Wadehra M, Iyer R, Goodglick L, Braun J. The tetraspan protein epithelial membrane protein-2 interacts with betal integrins and regulates adhesion. J Biol Chem. Oct. 25 2002;277(43):41094-41100.
43. Huang T, Fu M, Kuga D, et al. EMP2 regulates FAK signaling and contributes to pathogenesis of Glioblastoma Multiforme. submited. 2010.
44. Hochgrafe F, Zhang L, O'Toole S A, et al. Tyrosine phosphorylation profiling reveals the signaling network characteristics of Basal breast cancer cells. Cancer Res. Nov. 15 2010;70(22):9391-9401.
45. Schaller M D, Frisch S M. PND-1186 FAK inhibitor selectively promotes tumor cell apoptosis in three-dimensional environments. Cancer Biol Ther. May 2010;9(10):791-793.
46. Pylayeva Y, Gillen K M, Gerald W, Beggs H E, Reichardt L F, Giancotti F G. Ras- and PI3K-dependent breast tumorigenesis in mice and humans requires focal adhesion kinase signaling. J Clin Invest. Feb. 2009;119(2):252-266.
47. Dahlin K, Mager E M, Allen L, et al. Identification of genes differentially expressed in rat alveolar type I cells. Am J Respir Cell Mol Biol. September 2004;31(3):309-316.
48. Wadehra M, Sulur G G, Braun J, Gordon L K, Goodglick L. Epithelial membrane protein-2 is expressed in discrete anatomical regions of the eye. Exp Mol Pathol. April 2003;74(2):106-112.

The invention is now described with reference to the following examples. These examples are provided for the purpose of illustration only and the invention should in no way be construed as being limited to these examples but rather should be construed to encompass any and all variations which become evident as a result of the teachings provided herein.

Whereas, particular embodiments of the invention have been described herein for purposes of description, it will be appreciated by those skilled in the art that numerous variations of the details may be made without departing from the invention as described in the appended claims.

### EXAMPLES

### Example 1

### EMP2 Protein Expression in Cancerous Breast Tissue

A study was conducted to examine the expression level of EMP2 in breast cancer. Initially, a Western blot analysis and immunohistochemistry (IHC) of a set of 5 flash frozen human invasive ductal carcinoma samples and 3 samples of normal glandular breast epithelium was conducted.

In order to conduct this analysis breast cancer cell lines or tissue were lysed in Laemmli buffer, and for EMP2 detection, were treated with N-glycosidase F (New England-Biolabs, Beverly, Mass.) to remove N-link glycosylation. Proteins were separated using SDS-PAGE, transferred onto nitrocellulose membrane and blocked in 10% nonfat dry milk in TBS-Tween20 buffer. Blots were probed using rabbit anti-human EMP2 antisera (1:2000). Proteins were then detected by using a horseradish peroxidase conjugated goat anti-rabbit antibody. As a loading control, β-actin expression was detected using primary monoclonal anti-β actin (Sigma) and secondary horseradish peroxidase-conjugated goat anti-mouse IgG (Amersham™, Piscataway, N.J.). Bands were visualized using ECL™ detection reagents (Amersham™)

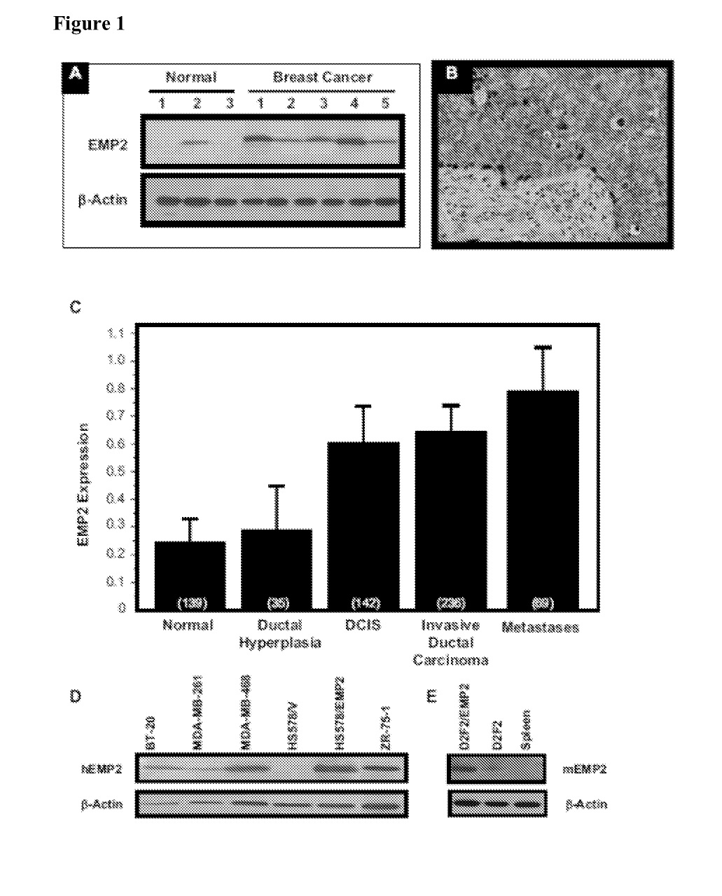

As shown in FIG. 1A, consistently, expression of EMP2 was robust in breast cancer samples compared to weak or non-detectable levels in non-malignant glandular epithelium. EMP2 was predominantly present at the membrane and/or cytoplasm of the malignant breast epithelium (FIG. 1B).

### Example 2

### Expression Profiled of EMP2 by Tissue Microarray

The expression profile of EMP2 in human breast cancer on a population basis was examined using tissue microarray (TMass.) technology. A high density breast cancer TMA was constructed using archival breast tissue samples from 212 patients who had breast surgery at the UCLA Medical Center between 1995 and 2000. Samples were collected as approved and monitored by the UCLA Institutional Review Board. The demographic, clinical, and pathology characteristics are shown in Table 2. Surgical samples from each patient often yielded more than one histology.

TABLE 2

| Clinical Parameter | n |
|---|---|
| Total Number of Patients | 74 |
| Age (≧55) | 32 |
| ER+ | 53 |
| PR+ | 52 |
| Her2+ | 18 |
| Invasive Ductal Carcinoma | 53 |
| Invasive Lobular Carcinoma | 4 |
| Other diagnosis | 17 |
| Grade 1 | 19 |
| Grade 2 | 22 |
| Grade 3 | 30 |
| Stage I | 26 |
| Stage II | 31 |
| Stage III | 16 |
| Stage IV | 1 |
| Tumor Size (≧2.5 cm) | 35 |
| Lymph node metastasis | 30 |
| Lympho-vasculature invasion | 24 |
| Recurrence | 8 |
| Disease-related death | 13 |

This study focused on the following categories: normal glandular/ductal epithelium (n=139 spots), ductal hyperplasia (DH; n=35 spots), ductal carcinoma in situ (DCIS; n=142 spots), invasive ductal carcinoma (IDC; n=236 spots), and lymph node metastatic lesions (n=69 spots).

A semiquantitative integrated scoring of the intensity and frequency of EMP2 staining was performed by a pathologist (RAS). The following formula was used to derive the integrated value: [3(% a) +2(% b) +1(% c]/100, where a, b, and c is the percentage of cells staining at intensity 3, 2, and 1, respectively.

Compared to non-malignant glandular and ductal mammary epithelium there was a significant increase in EMP2 expression in DCIS, invasive carcinoma and lymph node metastatic lesion (FIG. 1C).

On the TMA, there were 11 cases of triple negative breast cancer “TNBC.” TNBC tumors lack estrogen receptor (ER), progesterone receptor (PR) and Her-2/neu expression. Of these 11 cases, 8 (73%) had relatively high expression levels of EMP2 while 3 cases showed relatively lower levels of expression.

An additional set of 23 TNBC cases were also examined for EMP2 expression. Similar to above, 17 of these 23 cases of TNBC were positive for EMP2.

TABLE 3

| EMP2 | Number of |
|---|---|
| Expression | Cases |
| Below the level | 1 |
| of detection | |
| Weak | 5 |
| Strong | 17 |

### Example 3

### Effect of Anti-EMP2 Treatment In Vitro

To test the effects of anti-EMP2 treatment on breast cancer cells, the following breast cancer cell lines were utilized MDA-MB-231, MDA-MB-468, BT-20, and HS578, which are triple negative for ERa, PR and Her-2/neu expression and ZR-75-1, which expresses these three receptors.

Human breast cancer cell lines HS578 (a gift from Dr. John Colicelli, UCLA), BT-20, MDA-MB-231 (both gifts of Dr. Richard Pietras, UCLA), ZR-75-1 and MDA-MB-468 (American Type Culture Collection; ATCC, Manassas, Va.) were cultivated in DMEM medium (Mediatech, Manassas, Va.) supplemented with 10% fetal calf serum (Hyclone Laboratories, Logan, Utah), 2 mM L-glutamine, 1 mM sodium pyruvate, 100 U/ml penicillin, and 100 U/ml streptomycin (all from Invitrogen Life Technologies, Carlsbad, Calif.).

Cells were cultured at 37° C. in a humidified 5% CO2. Additionally, HS578 sublines were prepared which overexpress a EMP2 have been previously described. Control HS578 were also produced. These sublines were termed HS578/EMP2 and HS578/V, respectively. In addition, the murine mammary tumor D2F2 cells were utilized and maintained in RPMI medium supplemented as above. D2F2 cells (syngeneic for BALB/c mice) were a kind gift from Dr. Wei-Zen Wei (Wayne State University, Detroit, Mich.). Western blot analysis was used to confirm the EMP2 expression levels in each cell line

Each cell line was tested for EMP2 expression; a representative Western blot is shown in FIG. 1D. EMP2 is present in 4 out of 5 cell lines but below the level of detection in HS578 cells. A variant of HS578 which expressed EMP2 (HS578/EMP2) or, as a control, infected with vector plus GFP alone (HS578/V) was also constructed. The expression of EMP2 in HS578/EMP2 is shown in FIG. 1D. We also detected the expression of EMP2 in the murine mammary tumor cell line D2F2 and constructed a variant of D2F2 which overexpressed EMP2 coupled to a FLAG tag (D2F2/EMP2). Mouse spleenocytes was been used as a negative control (FIG. 1E).

For both in vitro and in vivo (described below) targeting a recombinant anti-EMP2 diabody which recognizes conserved regions of both human and murine EMP2 was utilized. In addition a full length humanized anti-EMP2 antibody (IgG1) was also constructed and utilized in these targeting experiments.

In order to construct the full length human anti-EMP2 antibody (IgG1) based on a previously described anti-EMP2 diabody construct, KS83. To accomplish this, the diabody variable (V) region sequence was obtained by PCR then cloned into pCR-II-TOPO vector (Invitrogen). This construct included a sequence encoding a function signal peptide for proper secretion. The cloning was confirmed by sequencing. The sequence encoding the EMP2 variable light (VL) and heavy chain regions (VH) were inserted into the κ light chain and γ heavy chain IgG1 expression vector, respectively. These vectors contain the cytomegalovirus promoter (CMV) and have been shown to secrete functional recombinant antibodies in murine myeloma cells. The heavy and light chain expression vectors were transfected into the murine myeloma cell line Sp2/0-Ag14 as described previously. Cells were subcloned then screened by ELISA (described below) using goat anti-human IgG (Invitrogen) and goat anti-human κ chain (Sigma-Aldrich). The five highest producing subclones were isolated to use for S-35 biosynthetic antibody labeling and immunoprecipitation with hyperimmune rabbit antihuman IgG (Sigma-Aldrich) and Staph A (IgGSorb, The Enzyme Center, Malden, Mass.), to validate and select the optimal clone. Once selected, cells were expanded into roller bottles to maximize the secretion of antibodies. After 2-3 weeks, supernatants were collected and filtered for purification as below.

Supernatants were passed over a 1.5 mL volume FlexColumn (Thermo Fisher Scientific) with 1 mL of protein A-Sepharose™ (Sigma-Aldrich), and bound proteins were eluted with 2 column volumes of 0.2 M citrate buffer (pH 4.5), 3 column volumes of 0.1 M glycine-HCl (pH 2.5) and 2 column volumes of 0.1 M glycine-HCl (pH 2.0), sequentially. The eluted fractions containing the desired antibodies were dialyzed against PBS with Slide-A-Lyzer® Dialysis Cassettes (Thermo Fisher Scientific). The final concentration of purified antibodies was measured with Nanodrop 2000 (Thermo Scientific). Binding specificity of the recombinant antibody was determined by ELISA and flow cytometry (see below).

For ELISA analysis biotinylated 24 amino acid peptides corresponding to the extracellular loop of human EMP2 were coated onto streptavidin-coated 96-well plates (Roche Applied Science, Indianapolis, IN). Specifically, bound antibodies were detected with HRP conjugated goat anti-human IgG (Jackson Immunoresearch, West Grove, PA) and TMB solution (eBioscience, San Diego, Calif.). Absorbance at 450 nm was determined using a microplate reader Model 550 (Bio-Rad, Hercules, Calif.).

For FACS analysis EMP2-positive cells (HEC1a/EMP2, HEC1a, or D2F2 cells) or EMP2-negative cells (EL4 or Ramos) were resuspended at a concentration of 10⁶ cells in 1 mL of cold PBS+0.2% BSA buffer (flow buffer). The cell suspension was centrifuged for 5 min at 500 g, at 4oC. Cells were then incubated with 1 μg of recombinant anti-EMP2 IgG1 for 2 h at 4° C. An IgG specific for the hapten dansyl, 5-dimethylamino naphthalene-l-sulfonyl chloride (DNS) was used as a non-targeted antibody negative control. Cells were washed three times and then incubated for 30 min at 4° C. with PE-conjugated goat anti-human IgG (Jackson Immunoresearch). Cells were washed and resuspended in flow buffer. Flow cytometry was immediately performed with a Becton Dickinson FACScan Analytic Flow Cytometer (Becton Dickinson) in the UCLA Jonsson Comprehensive Cancer Center and Center for AIDS Research Flow Cytometry Core Facility.

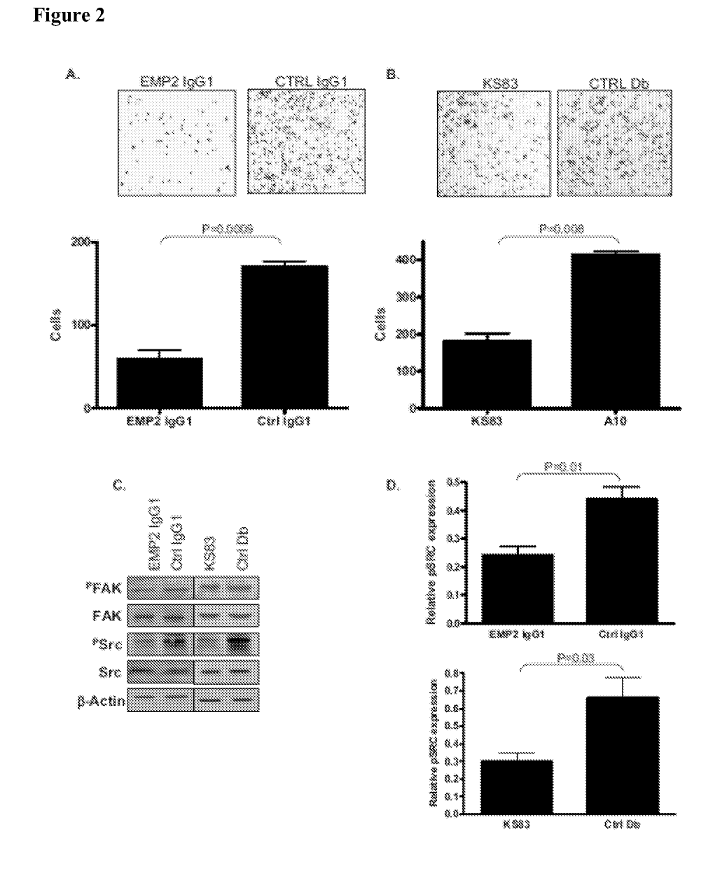

Previous studies have shown that EMP2 expression promotes invasion through FAK and Src activation. To determine if EMP2 antibodies inhibit invasion, cells were treated with anti-EMP2 KS83 or full length KS83-IgG1 or the appropriate controls. Two hour treatment with either EMP2 specific diabodies or full length KS83-IgG1 inhibited transwell invasion compared to the controls (FIG. 2A, B). Without being bound by theory, the apparent mechanism behind this action, is an EMP2 antibodies mediated reduced in Src phosphorylation (FIG. 2C, D). A modest reduction in FAK phosphorylation was also observed.

Although treatment of EMP2-expressing endometrial or ovarian cancer cells with the anti-EMP2 diabody results in cell death, such an effect has not been tested in breast cancer cells. In order to test cellular viability in the presence anti-EMP2 diabody KS83, control diabody A10, anti-EMP2 recombinant IgG1, or control IgG1 (Sigma-Aldrich), 5×10⁴ ZR-75-1, MDA-MB-468, HS578/EMP2, or HS578N cells were incubated in vitro with molar equivalent amounts of anti-EMP2 diabody (KS83), non-immune diabody (A10), full length KS83-IgG1, or a saline control for 24-96 hours. Cells were then harvested and the percentage of non-viable cells was determined using trypan blue exclusion..

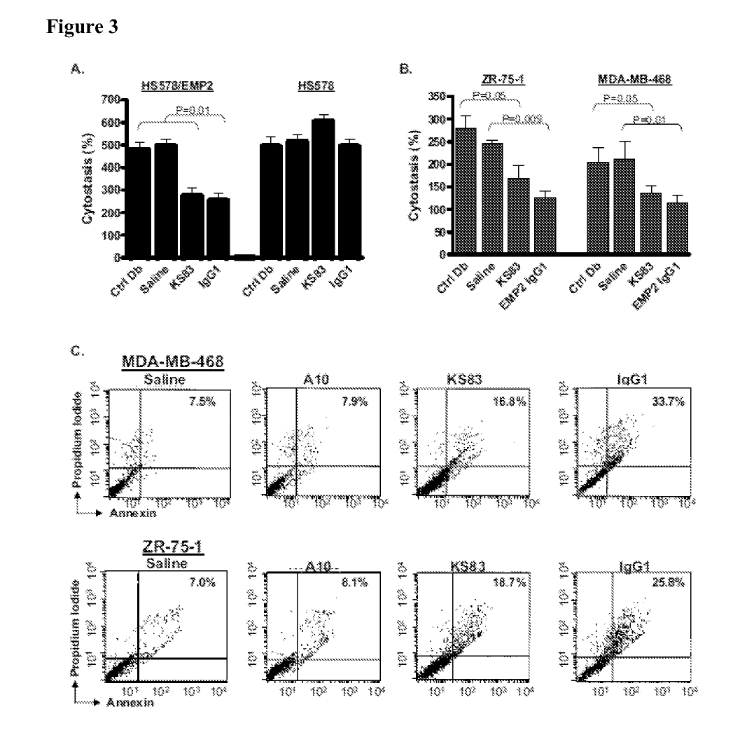

Treatment of ZR-75-1, MDA-MB-468, and HS578/EMP2 cells with either KS83 diabody or full length KS83-IgG1 resulted in ˜35%-50% loss of viability compared to the non-immune A10 diabody or saline control (FIG. 3A,B). Treatment of HS578N (EMP2-negative) cells with the diabodies or full length KS83-IgG1 caused no loss in viability indicating that the effect was dependent on EMP2 expression (FIG. 3A).

The cell death observed apparently occurs via an apoptotic mechanism. Cells incubated with KS83 diabody or full length KS83-IgG1 (or appropriate controls) were stained with the apoptosis marker, annexin V, as well as the viability marker propidium iodide. Cells were harvested and stained with an Annexin V-FITC detection kit according to manufacturer's instructions (BD Biosciences). As shown in FIG. 3B, incubation of ZR-75-1 or MDA-MB-468 cells with anti-EMP2 diabody or full length KS83-IgG1, induced an increase in apoptosis within 48 hours (FIG. 3C). Such an effect was not seen with control reagents.

### Example 4

### Effect of anti-EMP2 Treatment In Vivo

The effectiveness of anti-EMP2 immune reagents to limit tumor growth in a mouse model system was tested.

At the outset, the in vivo toxicity of KS83 diabodies and full length KS83 IgG₁ was assessed. 7-week-old female wild-type (C57BL/6) mice obtained from Jackson Laboratories. Mice were maintained in accordance with the National Academy of Science Guide for the Care and Use of Laboratory Animals in the Vivarium of UCLA. At least three animals per group were injected intravenously (i.v.) weekly with increasing concentrations (0.5-5 mg/kg) of anti-EMP2 IgG1 antibody or a vehicle control (sterile PBS) for 7 weeks. Serum was collected every 14 days. At the end of the time course, mice were euthanized by cervical dislocation. Tissues (kidney, liver, spleen, lung, uterus, heart, ovary, and skin) were collected fixed in formalin, processed, embedded in paraffin, sectioned, stained with Hematoxylin and Eosin, and analyzed for pathological changes by a pathologist. Complete blood counts and liver enzyme analysis (serum alanine aminotransferase, direct and total bilirubin) were quantified by the UCLA Medical Center Clinical Laboratories.

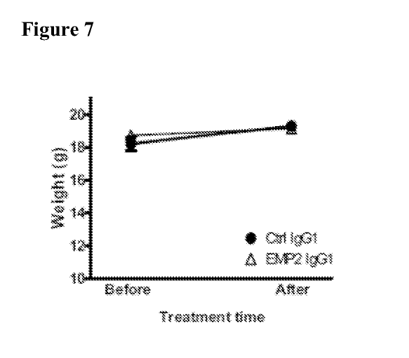

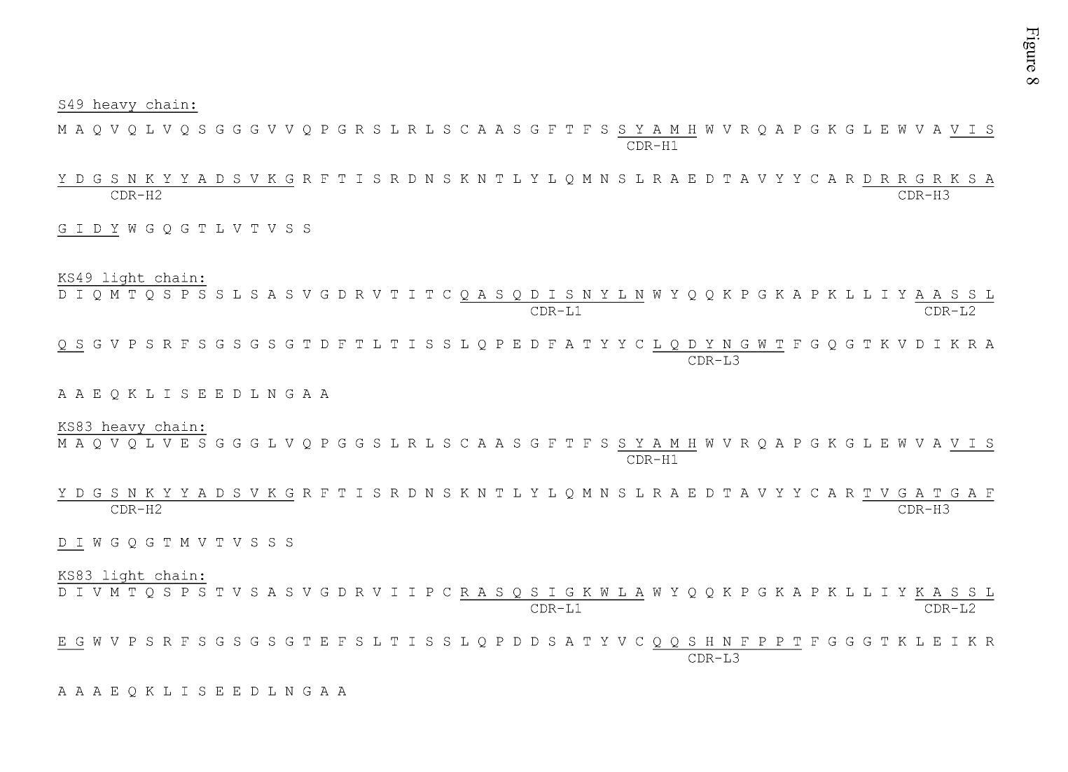

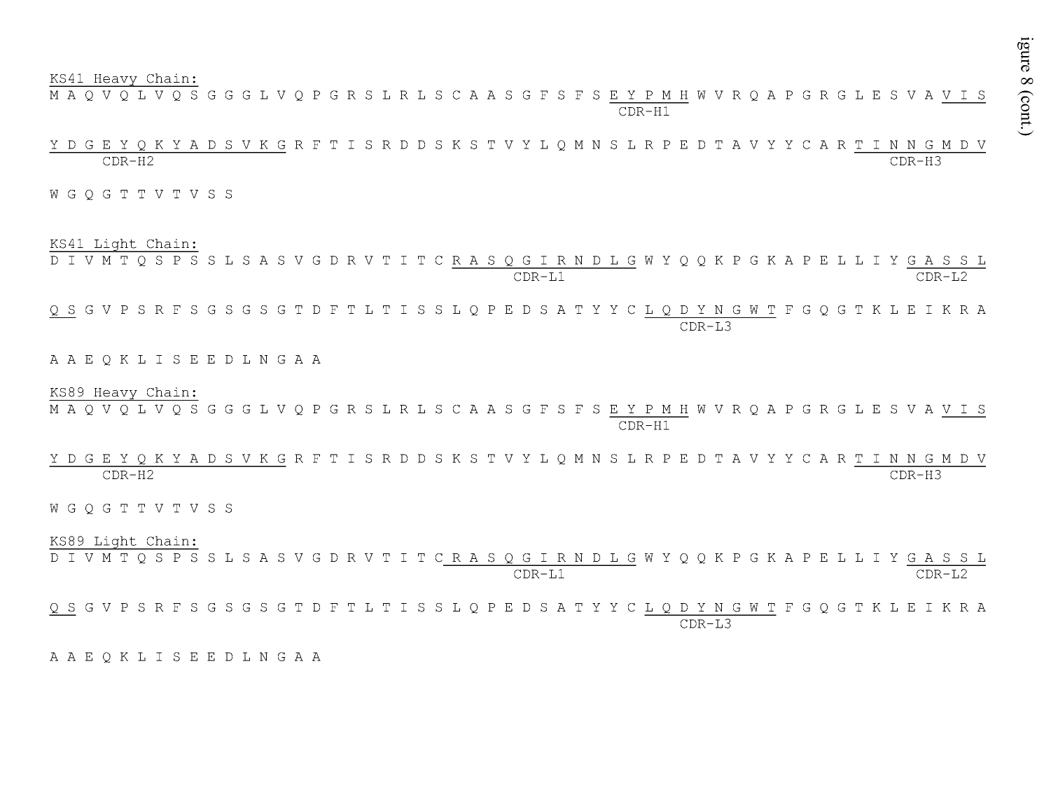

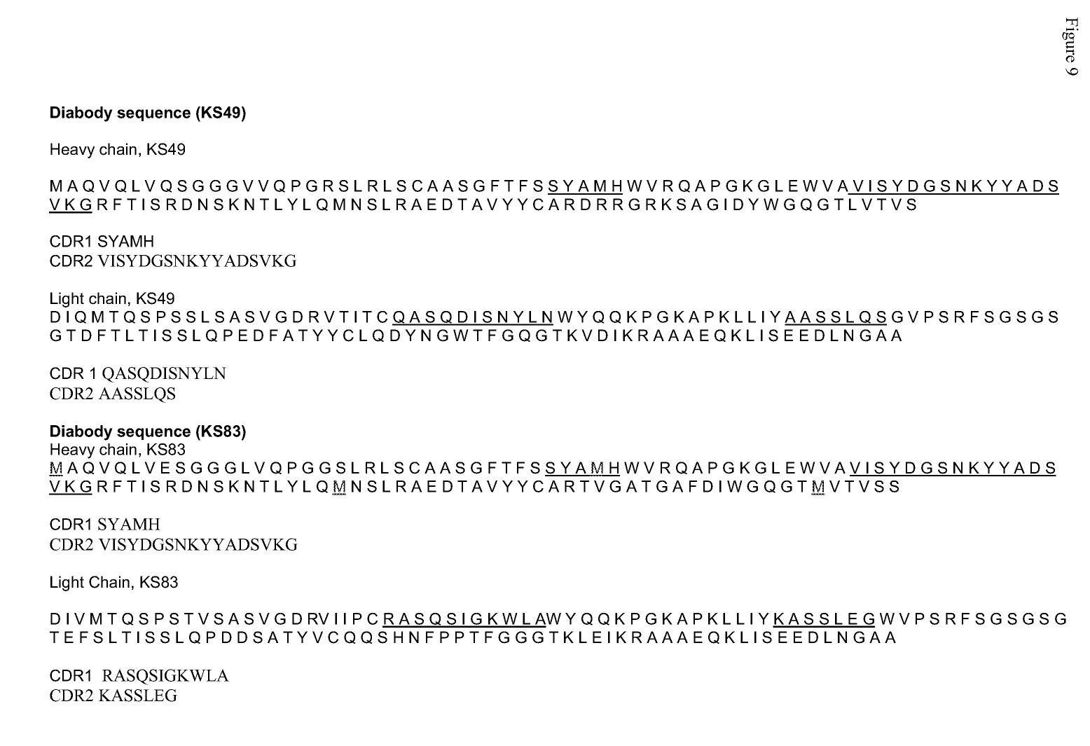

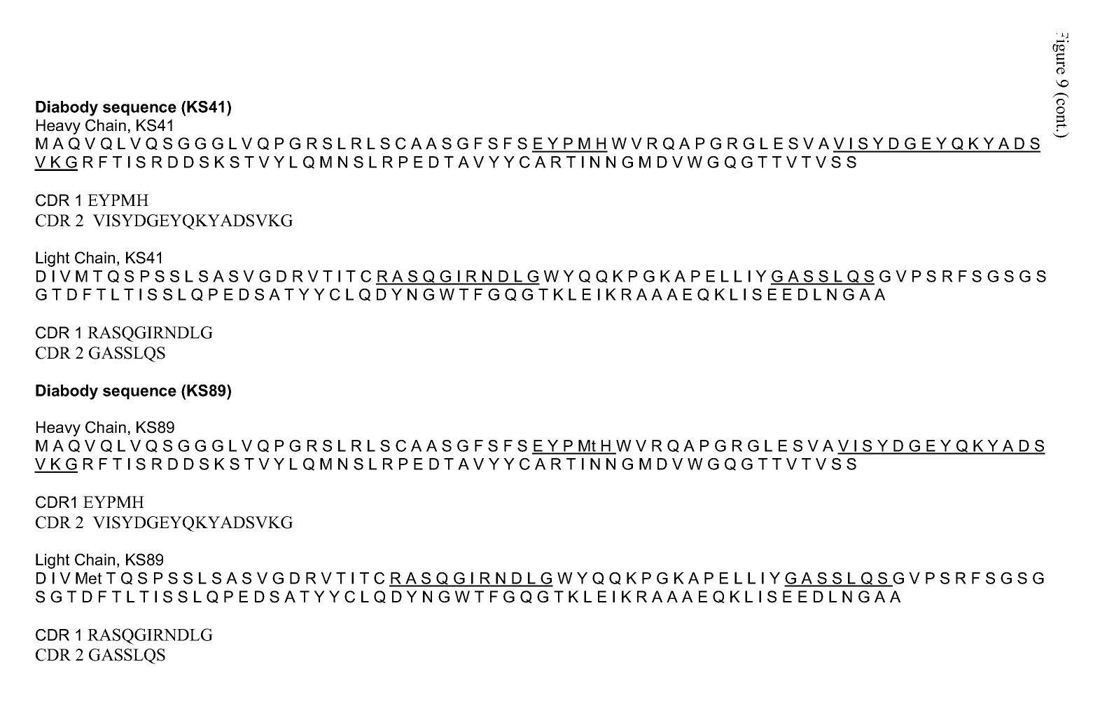

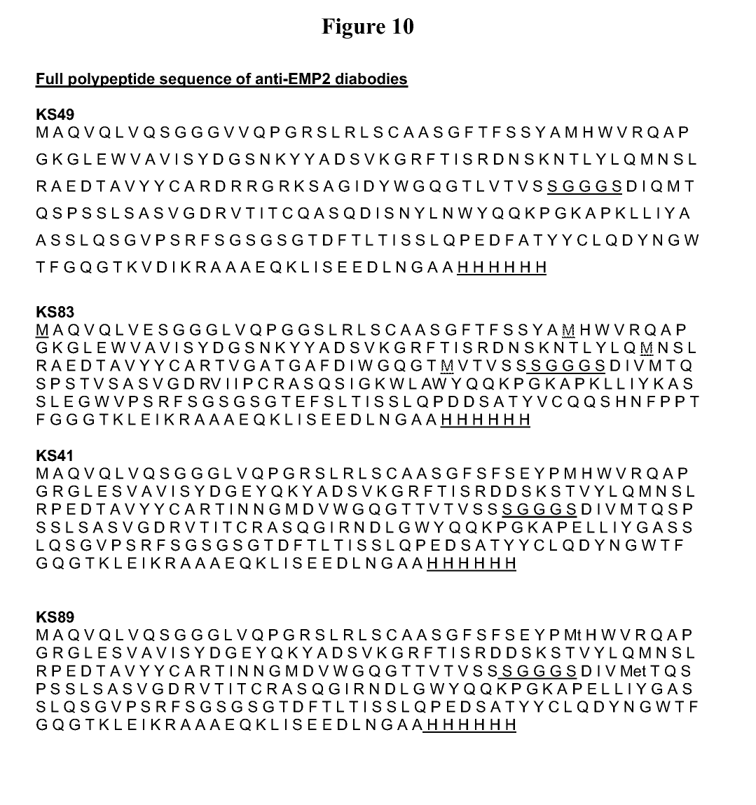

Weekly injections of KS83 or control diabodies demonstrated no detectable toxicity or adverse affects in mice. Likewise, upon extensive testing with twice weekly injections of the full length anti-EMP2 IgG1 in BALB/c mice (10 mg/kg) for seven weeks, no indication of systemic or tissue-specific damage or toxicity was observed (FIG. 7).

For a preclinical xenograft model of antibody directed therapy for breast cancer, the triple negative cell line, MDA-MB-468, and the anti-EMP2 diabody KS83 or the full-length anti-EMP2 IgG1 were utilized.

To create tumor xenograft models, 4-6-week old female BALB/c nude mice (Charles River, Mass.) were used for each condition. Mice were maintained in accordance with the National Academy of Science Guide for the Care and Use of Laboratory Animals in the vivarium of UCLA. Briefly, 5×106 MDA-MB-468 or 2×107 Ramos cells suspended in 5% BD matrigel™ (BD Biosciences) were injected subcutaneously (s.c.) into the shoulder of female athymic mice. Tumor volume was calculated with the formula: length×width/2.

When tumors became detectable (day 0), they were injected intratumor (i.t.) with 1 mg/kg dose of anti-EMP2 diabodies KS83 or non-immune diabody A10 twice a week. Alternatively, the tumors were injected with 3 mg/kg i.t. or between 1-10mg/kg systemically with anti-EMP2 IgG1 or control IgG (Sigma) weekly. Tumor size was monitored, and mice euthanized once tumors approached 1.5 cm in diameter or became ulcerated. Tumors were isolated, fixed and processed for hematoxylin and eosin staining

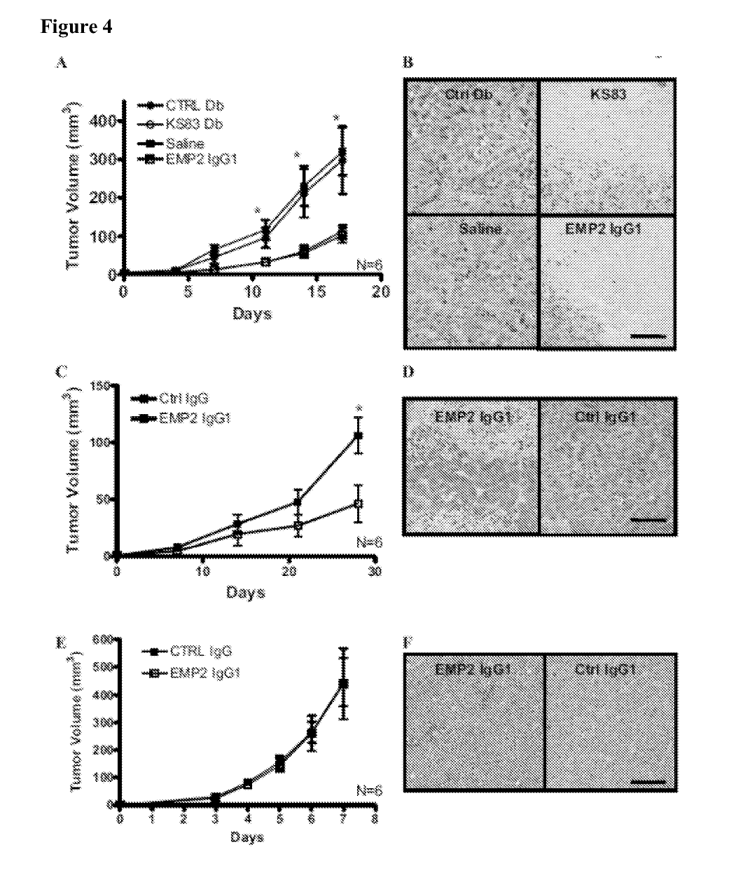

As shown in FIG. 4A, treatment of tumors with KS83 or the full length anti-EMP2 IgG1 resulted in significant reduction of tumor growth by day 15 compared to non-immune reagent treatment. Upon histological examination, tumors treated with anti-EMP2 reagents showed wide areas of necrosis in contrast with tumors treated with non-immune reagents (FIG. 4B).

Systemic injections of anti-EMP2 IgG1 as a therapeutic approach was also tested. Similar to i.t. injection, systemic treatment with full length anti-EMP2 IgG1 significantly reduced tumor growth compared to treatment with control IgG (FIG. 4C; day 28). This model also produced extensive necrosis in response to anti-EMP2 IgG treatment (FIG. 4D). Comparatively, reduced areas of necrosis were observed in the control IgG treated group compared with the anti-EMP2 IgG1 treated group. To validate that the effects of anti-EMP2 IgG1 treatment were specific, the B lymphoma cell line Ramos was used as a control. Ramos cells do not express EMP2. Injections twice a week of either anti-EMP2 IgG1 or Ctrl IgG1 showed no difference in tumor load (FIG. 4E) or in tumor histology (FIG. 4F).

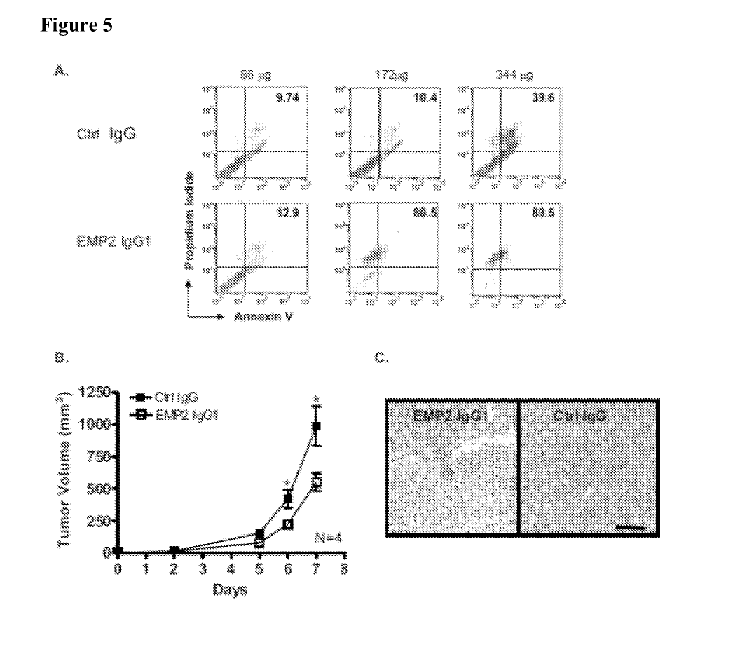

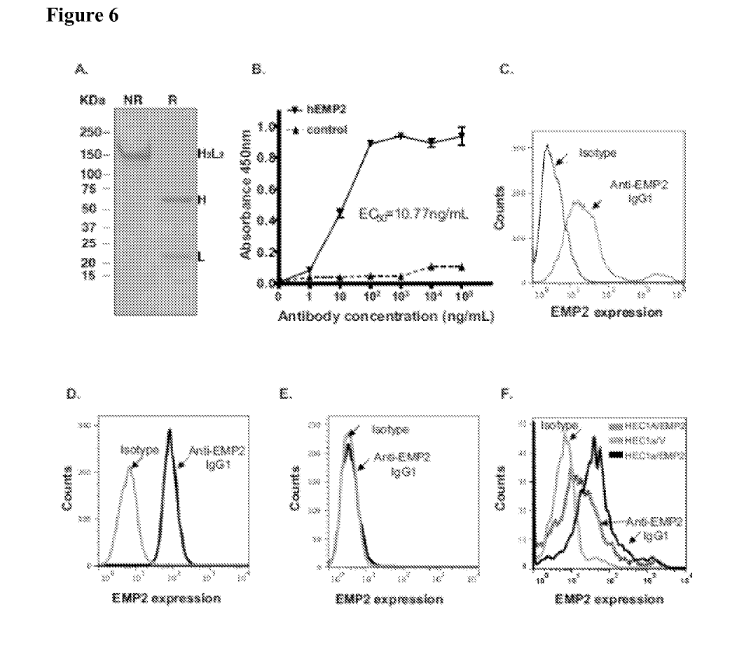

As a further confirmation of the efficacy of anti-EMP2 IgG1 treatment, murine mammary D2F2 cells were utilized. D2F2 cells express EMP2 on the plasma membrane and are syngeneic to BALB/c mice. Susceptibility of D2F2 cells to anti-EMP2 IgG1 treatment was determined through titration of antibody and measurement of apoptosis and cell death by flow cytometry (FIG. 5A). A dose dependent increase in cell death relative to antibody concentration was observed, and the effects were significantly increased compared to treatment with the control IgG.

To determine if this effect could be recapitulated in vivo, 1×10⁵ D2F2 tumors were injected into the mammary pad of BALB/c mice (Charles River). Once tumors were detectable, they were treated twice with 10 mg/kg of anti-EMP2 IgG1 or control IgG. After the final point, mice were euthanized and tumors isolated as above. While these tumors grow very rapidly, two treatments within the week of anti-EMP2 IgG1 significantly reduced tumor load by 50% compared to control IgG treatment (FIG. 5B) with tumors exhibiting pronounced necrosis (FIG. 5C).

For statistical analysis, differences in in vitro phenotypic changes or in vivo tumor growth were evaluated using a two-tailed Student's unpaired t-test at a 95% confidence level (GraphPad Prism version 3.0; GraphPad Software, La Jolla, Calif.). P values<0.05 were considered significant. TMA analyses were performed as previously described (30-34) with the Mann-Whitney test for two-group comparisons. A P value<0.05 was considered significant.

Each publication, patent application, patent, and other reference cited herein is incorporated by reference in its entirety to the extent that it is not inconsistent with the present disclosure. In particular, all publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A method of treating a patient for breast cancer, wherein the breast cancer comprises a triple negative breast cancer tumor, the method comprising administering to the patient an effective amount of an antibody wherein the antibody specifically binds to an epitope in the second extracellular loop of EMP2, wherein the epitope comprises the amino acid sequence DIHDKNAKFYPVTREGSYG.

2. The method of claim 1, wherein the antibody further comprises a physiological acceptable carrier or a pharmaceutically acceptable carrier.

3. The method of claim 1, wherein the antibody competes with an antibody comprising the heavy and light chain variable regions of a KS49, a KS41, a KS83, or a KS89 diabody.

4. The method of claim 1, wherein the antibody shares 90% amino acid identity with heavy and light chain variable regions of a KS49, a KS41, a KS83, or a KS89 diabody.

5. The method of claim 1, wherein the antibody comprises CDR sequences identical to those of a KS49, a KS41, a KS83, or a KS89 diabody.

6. The method of any one of claims 1-3, further comprising administering to the patient an effective amount of at least one additional anti-cancer agent.

7. The method of claim 6, wherein the at least one additional anti-cancer agent is selected from the group consisting of platinum-based chemotherapy drugs, taxanes, tyrosine kinase inhibitors, anti-EGFR antibodies, anti-ErbB2 antibodies, and combinations thereof.

8. The method of claim 6, wherein the at least one additional anti-cancer agent comprises an EGFR inhibitor.

9. The method of claim 8, wherein the EGFR inhibitor comprises an anti-EGFR antibody.

10. The method of claim 9, wherein the anti-EGFR antibody comprises cetuximab.

11. The method of claim 9, wherein the anti-EGFR antibody is selected from the group consisting of matuzumab, panitumumab, and nimotuzumab.

12. The method of claim 6, wherein the EGFR inhibitor is a small molecule inhibitor of EGFR signaling.

13. The method of claim 12, wherein the small molecule inhibitor of EGFR signaling is selected from the group consisting of gefitinib, lapatinib, canertinib, pelitinib, erlotinib HCL, PKI-166, PD158780, and AG 1478.

14. The method of claim 6, wherein the at least one additional anti-cancer agent comprises a VEGF inhibitor.

15. The method of claim 14, wherein the VEGF inhibitor comprises an anti-VEGF antibody.

16. The method of claim 15, wherein the anti-VEGF antibody is bevacizumab.

17. The method of any of claims 1-16 wherein the antibody is conjugated with an effector moiety.

18. The method of claim 17, wherein the effector moiety is a toxic agent.

19. The method of claim 18, wherein the toxic agent is such as ricin.

20. The method of any of claims 1-19, wherein the treatment comprises blocking invasiveness of the cancer.

21. The method of any of claims 1-20, wherein the antibodies are used in vaccine therapies for the cancer.

22. The method of any of claims 1-21, wherein the patient is human or mammal.

23. The method of any one of claims 1-22, further comprising a companion diagnostic.

24. The method of claim 23, wherein the companion diagnostic comprises an anti-EMP2 antibody.
